(19) United States
(12) Patent Application Publication
(10) Pub. No.: US 2026/0335346 A1
(43) Pub. Date: Oct. 29, 2026

(54) PREBIOTICS FOR TREATING AND/OR PREVENTING VITAMIN K2 DEFICIENCY

(71) Applicant: SOCIÉTÉ DES PRODUITS NESTLÉ S.A., Vevey (CH)

(72) Inventors: Claire Laurence Lucie Marie BOULANGE, Oron-la-Ville (CH); Stéphane DUBOUX, St-Prex (CH); Esther CAMPOS GIMENEZ, Billens (CH); Marie Noëlle HORCAJADA, Echenevex (FR); Nicolas BONNET, Beaumont (FR)

(21) Appl. No.: 19/486,301

(22) PCT Filed: May 24, 2024
(86) PCT No.: PCT/EP2024/064337

§ 371 Date: Nov. 20, 2025

(30) Foreign Application Priority Data
May 25, 2023 (EP) 23175289.0

(51) Int. Cl. A61K 31/702; A23L 33/00; A61K 31/07; A61K 31/122; A61K 31/592; A61K 31/593; A61K 31/716; A61K 35/00; A61K 35/745; A61K 35/747; A61P 1/00; A61P 19/00

(52) U.S. Cl. CPC A61K 31/702; A23L 33/40; A61K 31/07; A61K 31/122; A61K 31/592; A61K 31/593; A61K 31/716; A61K 35/745; A61K 35/747; A61P 1/00; A61P 19/00; A61K 2035/115

(57) ABSTRACT

The present invention relates to a prebiotic agent for use in treating and/or preventing vitamin K2 deficiency in a subject. The present invention relates to the use of a prebiotic agent to promote vitamin K2 production in the gut of a subject.

### FIELD OF THE INVENTION

The present invention relates to agents, compositions, and methods for treating and/or preventing vitamin K2 deficiency in a subject. The present invention also relates to agents, compositions, and methods for promoting vitamin K2 production in the gut of a subject.

### BACKGROUND TO THE INVENTION

Vitamin K2 activates vitamin K-dependent proteins that support many biological functions, such as bone mineralization, the inhibition of vascular stiffness, the improvement of endothelial function, the maintenance of strong teeth, brain development, joint health, and optimal body weight (Kozioł-Kozakowska, A. and Maresz, K., 2022. Children, 9(1), p. 78).

However, over the last few decades, vitamin K2 intakes among parents and their offspring have decreased significantly, resulting in serious health implications. Moreover, secondary vitamin K2 deficiency can occur in people who consume adequate amounts, but have malabsorption conditions or liver disease, when vitamin K antagonist drugs are used, or following long-term use of antibiotics of glucocorticoids.

Thus, there is a demand for new nutritional interventions to treat and/or prevent vitamin K2 deficiency.

### SUMMARY OF THE INVENTION

The present inventors have surprisingly found that vitamin K2 production in the gastrointestinal tract (e.g. via conversion of vitamin K1) is promoted by a prebiotic agent, for example comprising bovine milk oligosaccharides (BMOs), human milk oligosaccharides (HMOs), and/or cello-oligosaccharides (COS).

In one aspect, the present invention provides a prebiotic agent for use in treating and/or preventing vitamin K2 deficiency in a subject.

In another aspect, the present invention provides use of a prebiotic agent in the manufacture of a medicament for treating and/or preventing vitamin K2 deficiency in a subject.

In another aspect, the present invention provides a method for treating and/or preventing vitamin K2 deficiency in a subject in need thereof, the method comprising administering to the subject an effective amount of a prebiotic agent.

The prebiotic agent may treat or prevent vitamin K2 deficiency by promoting vitamin K2 production in the subject's gut. For example, the prebiotic agent may treat or prevent vitamin K2 deficiency by promoting de novo menaquinone-7 production and/or bioconversion from phylloquinone to menaquinone-4 in the subject's gut.

In another aspect, the present invention provides use of a prebiotic agent to promote vitamin K2 production in the gut of a subject.

In another aspect, the present invention provides a method for promoting vitamin K2 production in the gut of a subject, the method comprising administering to the subject an effective amount of a prebiotic agent.

The prebiotic agent may be any suitable prebiotic agent that promotes vitamin K2 production in the subject's gut. Suitably, the prebiotic agent is selected from one or more of: bovine milk oligosaccharides (BMOs), human milk oligosaccharides (HMOs), cellobiose, cello-oligosaccharides (COS), inulin, lactose, fructo-oligosaccharides (FOS), galacto-oligosaccharides (GOS), and β-glucan. In some embodiments, the prebiotic agent is selected from one or more of: bovine milk oligosaccharides (BMOs), a human milk oligosaccharide (HMO) mixture, and cello-oligosaccharides (COS).

In some embodiments, the prebiotic agent comprises BMOs in an amount of from about 80 wt % to about 100 wt %, with respect to the total weight of the prebiotic agent.

In some embodiments, the prebiotic agent comprises or consists of one or more HMO. In some embodiments, the prebiotic agent comprises or consists of at least one sialylated oligosaccharide, at least one fucosylated oligosaccharide, and/or at least one N-acetylated oligosaccharide. Suitably, the at least one sialylated oligosaccharide is selected from the group consisting of 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof. In some embodiments, the at least one sialylated oligosaccharide is selected from 3′-sialyllactose (3′-SL), 6′-sialyllactose (6′-SL) and combinations thereof. Suitably, the at least one fucosylated oligosaccharide is selected from the group consisting of 2′-fucosyllactose (2′FL), 3-fucosyllactose (3FL), difucosyllactose (diFL), lacto-N-fucopentaose-I (LNFP-I), lacto-N-fucopentaose-II (LNFP-II), lacto-N-fucopentaose-III (LNFP-III), lacto-N-fucopentaose-V (LNFP-V), lacto-neofucopentaose V (LNnFP-V), lacto-N-difucosylhexaose-I (LNDFH-1), lacto-N-neodifucosylhexaose (LNnDFH), monofucosyllacto-n-hexaose-III (MFNLH-III) difucosyllacto-N-hexaose-a (DFLNHa) and combinations thereof. In some embodiments, the at least one fucosylated oligosaccharide is 2′-fucosyllactose (2′FL) and/or difucosyllactose (diFL). Suitably, the at least one N-acetylated oligosaccharide is selected from the group consisting of N-acetyl-glucosamine, N-acetyl-galactosamines, lacto-N-tetraose (LNT), lacto-N-neotetraose (LNnT), and combinations thereof. In some embodiments, the at least one N-acetylated oligosaccharide is selected from lacto-N-tetraose (LNT), lacto-N-neotetraose (LNnT) and combinations thereof.

In some embodiments, the prebiotic agent comprises or consists of cello-oligosaccharides (COS).

The prebiotic agent may be administered to the subject in any suitable amounts. Suitably, the prebiotic agent is administered to the subject in a total amount of from about 0.5 g/day to about 10 g/day. Suitably, BMOs are administered to the subject in a total amount of from about 0.5 g/day to about 10 g/day. Suitably, HMOs are administered to the subject in a total amount of from about 0.5 g/day to about 10 g/day. Suitably, cello-oligosaccharides (COS) are administered to the subject in a total amount of from about 0.5 g/day to about 10 g/day.

The prebiotic agent may be administered in combination with vitamin K1. Vitamin K2 may be formed via metabolic conversion of vitamin K1 during its absorption in the intestinal mucosa and in other organs. The prebiotic agent and vitamin K1 may be administered separately, simultaneously or sequentially. In preferred embodiments, the prebiotic agent and vitamin K1 are administered simultaneously. Suitably, vitamin K1 is administered to the subject in an amount of from about 5 μg/day to about 200 μg/day.

The prebiotic agent may be administered in combination with a probiotic agent. The present inventors have surprisingly found that the production of vitamin K2 in the gastrointestinal tract (e.g. via conversion of vitamin K1) can be further promoted by administration of a probiotic agent. The prebiotic agent and the probiotic agent may be administered separately, simultaneously or sequentially. In preferred embodiments, the prebiotic agent and the probiotic agent are administered simultaneously. The probiotic agent may comprise any suitable probiotic. Suitably, the probiotic agent comprises Lactobacillus rhamnosus, Bifidobacterium infantis and/or Bifidobacterium lactis. Suitably, a probiotic agent is administered to the subject in a total amount of from about 10⁶ cfu/day to about 10¹² cfu/day.

The prebiotic agent (or combination therewith) may be provided in any suitable form, for example in the form of a composition. The prebiotic agent (or combination therewith) may be provided in the form of a nutritional composition. The prebiotic agent (or combination therewith) may be provided in the form of a medical food product for clinical nutrition.

Suitably, the composition comprises the prebiotic agent in a total amount of from about 0.5 g/100 g to about 10 g/100 g, on a dry weight basis. Suitably, the composition comprises vitamin K1 in an amount of about 5 μg/100 g to about 200 μg/100 g, on a dry weight basis. Suitably, the composition comprises a probiotic agent in an amount of about 10⁶ cfu/100 g to about 10¹² cfu/100 g, on a dry weight basis.

The subject may be any suitable subject. Suitably, the subject is a human or an animal. In preferred embodiments, the subject is a human. In some embodiments, the subject is a juvenile, an adolescent, a child, a toddler, or an infant. In some embodiments, the subject is a child, a toddler, or an infant. In other embodiments, the subject is an adult. The subject may have or may be at risk of vitamin K2 deficiency. In some embodiments, the subject has or is at risk of dyslipidemia, diabetes, thalassemia major (TM), cystic fibrosis (CF), inflammatory bowel diseases (IBD), or chronic liver diseases. In some embodiments, the subject has or is at risk of reduced bone growth and/or bone strength.

### DESCRIPTION OF DRAWINGS

FIG. 1—Effect of human milk oligosaccharides (HMOs) on vitamin K2 production in a gut model

The following groups were evaluated: Blank; Single HMO (1.3 g/L 2FL); HMO mix (2.5 g/L 2FL, DiFL, LNnT, LNT, and 6SL); B. infantis (10⁷ cfu/ml); Single HMO+B. infantis; HMO mix+B. infantis.

FIG. 2—Effect of oligosaccharides and probiotic agents on vitamin K2 production in a gut model

(A) The following groups were evaluated (in the absence of milk matrix): Blank; and HMOs+BMOs (7.2 g/L total). (B) The following groups were evaluated: Blank; MM (a milk matrix comprising a mix of 6 HMOs); MM+B. infantis (10¹¹ cfu/g).

FIG. 3—Effect of human milk oligosaccharides (HMOs) on vitamin K2 production in a gut model

(A) The following groups were evaluated (in a milk matrix in the absence of L. rhamnosus LPR): Blank; COS (2700 mg/L); COS (2025 mg/L)+B-glucan (675 mg/L); and COS (2025 mg/L)+β-glucan (675 mg/L)+B. lactis (4.5×10⁶ cfu/ml). (B) The following groups were evaluated (in a milk matrix in the presence of 4.5×10⁷ cfu/ml L. rhamnosus LPR): Blank; COS (2700 mg/L); COS (2025 mg/L)+β-glucan (675 mg/L); and COS (2025 mg/L)+β-glucan (675 mg/L)+B. lactis (4.5×10⁶ cfu/ml). (C) The following groups were evaluated (in a milk matrix): w/o LPR (no L. rhamnosus LPR added); and w/LPR (4.5×10⁷ cfu/ml L. rhamnosus LPR).

FIG. 4—Preclinical experimental design Faltering growth group (with vit.K1): the number of pups per BALB/c mother has been increased by 50% from D8 to D18 to induce a food restriction. In both groups (normal and faltering growth), weaning happened at D18. Male and female mice then received food ad libitum and a daily nutritional supplementation through pipet feeding for 30 days.

FIGS. 5A&B—Impact of Vit.K2AD on trabecular BV/TV and cortical BMD assessed through micro-computed tomography on femurs.

Micro-computed tomography (μCT UCT35, Scanco Medical AG, Basserdorf, Switzerland) was used to assess trabecular and cortical microstructure, respectively, at distal metaphysis and midshaft diaphysis of femurs, as previously described1. Briefly, trabecular and cortical bone regions were evaluated using isotropic 6 μm voxels. For the femoral trabecular region, to eliminate the primary spongiosa, 30 slices of bone under the distal growth plate were not considered. The 80 slices of secondary spongiosa directly below were analyzed. Femoral cortical structure was assessed using 60 continuous CT slides located at the femur midshaft. Morphometric variables were computed from binarized images using direct, three-dimensional techniques that do not rely on prior assumptions about the underlying structure2. For the trabecular bone regions, BV/TV fraction (%) was assessed. For cortical bone at the femoral midshaft, cortical bone mineral density (Ct.BMD) was assessed.

FIGS. 6A&B—Impact of Vit.K2AD on femur strength assessed through a 3-point bending test

A 3-point bending test was performed to test the biomechanical properties of the femur as previously described (C. H. Turner, D. B. Burr, Basic biomechanical measurements of bone: a tutorial, Bone, 1993, 14 (4): 595-608). The load was applied in a compression mode at a nominal deformation rate of 0.08 mm/s until fracture. Load-displacement curves were recorded to determine the force at rupture normalized by the antero-posterior diameter of the femur (Fmax (N)/AP (mm)) and the energy at rupture normalized by the antero-posterior diameter of the femur (Wmax (N)/AP (mm)).

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

Any reference to prior art documents in this specification is not to be considered an admission that such prior art is widely known or forms part of the common general knowledge in the field. All publications mentioned in the specification are herein incorporated by reference.

As used in this specification, the words “comprises”, “comprising”, and similar words, are not to be interpreted in an exclusive or exhaustive sense. In other words, they are intended to mean “including, but not limited to”. The terms “comprises”, “comprising”, and similar words also include the term “consisting of”.

The practice of the present invention will employ, unless otherwise indicated, conventional techniques which are within the capabilities of one of ordinary skill in the art. Such techniques are explained in the literature. Unless defined otherwise, all technical and scientific terms used herein have the same meaning as commonly understood by one of ordinary skill in the art.

Numeric ranges are inclusive of the numbers defining the range and all percentages disclosed herein are on a w/w basis, unless stated otherwise. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

### Prebiotics

The present invention provides a prebiotic agent for use in treating and/or preventing vitamin K2 deficiency in a subject.

As used herein, the term “prebiotic” may refer to a non-digestible component that benefits the subject by selectively stimulating the favourable growth and/or activity of one or more microbial taxa. The prebiotic agent may be any suitable prebiotic agent that promotes vitamin K2 production. Exemplary prebiotics include bovine milk oligosaccharides (BMOs), human milk oligosaccharides (HMOs), cellobiose, cello-oligosaccharides (COS), inulin, lactose, fructo-oligosaccharides (FOS), galacto-oligosaccharides (GOS), and β-glucan.

In some embodiments, the prebiotic agent is selected from one or more of: bovine milk oligosaccharides (BMOs), human milk oligosaccharides (HMOs), cellobiose, cello-oligosaccharides s (COS), inulin, lactose, fructo-oligosaccharides (FOS), galacto-oligosaccharides (GOS), and β-glucan.

In some embodiments, the prebiotic agent is selected from one or more of: bovine milk oligosaccharides (BMOs), a human milk oligosaccharide (HMO) mixture, and cello-oligosaccharides (COS). In some embodiments, the prebiotic agent comprises BMOs and HMOs. In some embodiments, the prebiotic agent comprises HMOs. In some embodiments, the prebiotic agent comprises cello-oligosaccharides (COS).

### Bovine Milk Oligosaccharides

The prebiotic agent used in the present invention may comprise or consist of bovine milk oligosaccharides (BMOs).

Oligosaccharides in bovine milk are assembled in the mammary gland by combining the monosaccharides glucose (Glc), galactose (Gal), N-acetylglucosamine (GlcNAc), N-acetylgalactosamine, fucose, and the sialic acids N-acetylneuraminic acid and N-glycolylneuraminic acid. The collection of BMOs found in milk and colostrum has been extensively profiled, with 30-50 structures typically being identified in comprehensive studies (see e.g. Robinson, R. C., 2019. Frontiers in nutrition, 6, p. 50).

Although bovine milk typically contains fewer oligosaccharide structures than human milk, the two share at least ten common structures, including 3′-sialyllactose and 6′-sialyllactose, which comprise a large percentage of the BMO pool (see e.g. Robinson, R. C., 2019. Frontiers in nutrition, 6, p. 50).

The prebiotic agent may comprise BMOs in an amount of about 50 wt % or more, about 60 wt % or more, about 70 wt % or more, about 80 wt % or more, about 85 wt % or more, about 90 wt % or more, about 95 wt % or more, or about 100 wt %, with respect to the total weight of the prebiotic agent.

### Human Milk Oligosaccharides (HMOs)

The prebiotic agent used in the present invention may comprise or consist of one or more human milk oligosaccharide (HMO).

Many different kinds of HMOs are found in the human milk and are typically based on a combination of glucose, galactose, sialic acid (N-acetylneuraminic acid), fucose and/or N-acetylglucosamine with many and varied linkages between them. Almost all HMOs have a lactose moiety at their reducing end while sialic acid and/or fucose (when present) occupy terminal positions at the non-reducing ends. HMOs can be acidic (e.g. charged sialic acid containing oligosaccharides) or neutral (e.g. fucosylated oligosaccharides).

Suitably, the one or more HMO may comprise at least one fucosylated oligosaccharide, at least one sialylated oligosaccharide, and/or at least one N-acetylated oligosaccharide. In some embodiments, the one or more HMO comprises or consists of at least one fucosylated oligosaccharide, at least one sialylated oligosaccharide, and at least one N-acetylated oligosaccharide. In some embodiments, the one or more HMO comprises or consists of 2′-fucosyllactose (2′FL), difucosyllactose (diFL), 6′-sialyllactose (6′-SL), lacto-N-tetraose (LNT), and lacto-N-neotetraose (LNnT). In some embodiments, the one or more HMO comprises or consists of 2′-fucosyllactose (2′FL), difucosyllactose (diFL), 6′-sialyllactose (6′-SL), 3′-sialyllactose (3′-SL), lacto-N-tetraose (LNT), and lacto-N-neotetraose (LNnT).

In some embodiments (e.g. when the prebiotic agent comprises BMOs), the prebiotic agent comprises about 0.5 wt % to about 2 wt %, with respect to the total weight of the prebiotic agent, of at least one fucosylated oligosaccharide. In some embodiments (e.g. when the prebiotic agent comprises BMOs), the prebiotic agent comprises about 2 wt % to about 6 wt %, with respect to the total weight of the prebiotic agent, of at least one sialylated oligosaccharide. In some embodiments (e.g. when the prebiotic agent comprises BMOs), the prebiotic agent comprises about 1 wt % to about 4 wt %, with respect to the total weight of the prebiotic agent, of at least one N-acetylated oligosaccharide.

In some embodiments (e.g. when the prebiotic agent comprises BMOs), the prebiotic agent comprises about 2 wt % to about 6 wt %, with respect to the total weight of the prebiotic agent, of at least one fucosylated oligosaccharide; and about 1 wt % to about 4 wt %, with respect to the total weight of the prebiotic agent, of at least one N-acetylated oligosaccharide.

In some embodiments (e.g. when the prebiotic agent comprises BMOs), the prebiotic agent comprises: about 2 wt % to about 6 wt %, with respect to the total weight of the prebiotic agent, of at least one fucosylated oligosaccharide; about 0.5 wt % to about 2 wt %, with respect to the total weight of the prebiotic agent, of at least one sialylated oligosaccharide; and about 1 wt % to about 4 wt %, with respect to the total weight of the prebiotic agent, of at least one N-acetylated oligosaccharide.

In other embodiments (e.g. when the prebiotic agent does not comprise BMOs), the prebiotic agent comprises about 30 to about 80 wt %, about 40 to about 80 wt %, or about 50 to about 70 wt %, with respect to the total weight of the prebiotic agent, of at least one fucosylated oligosaccharide. In some embodiments (e.g. when the prebiotic agent does not comprise BMOs), the prebiotic agent comprises about 10 to about 35 wt %, about 10 to about 30 wt %, or about 10 to about 25 wt %, with respect to the total weight of the prebiotic agent, of at least one sialylated oligosaccharide. In some embodiments (e.g. when the prebiotic agent does not comprise BMOs), the prebiotic agent comprises about 10 to about 35 wt %, about 15 to about 30 wt %, or about 15 to about 20 wt %, with respect to the total weight of the prebiotic agent, of at least one N-acetylated oligosaccharide.

In some embodiments (e.g. when the prebiotic agent does not comprise BMOs), the prebiotic agent comprises or consists of about 30 to about 80 wt %, about 40 to about 80 wt %, or about 50 to about 70 wt %, with respect to the total weight of prebiotic agent, of at least one fucosylated oligosaccharide; and about 10 to about 35 wt %, about 15 to about 30 wt %, or about 15 to about 20 wt %, with respect to the total weight of the prebiotic agent, of at least one N-acetylated oligosaccharide.

In some embodiments (e.g. when the prebiotic agent does not comprise BMOs), prebiotic agent comprises or consists of: about 30 to about 80 wt %, about 40 to about 80 wt %, or about 50 to about 70 wt %, with respect to the total weight of the prebiotic agent, of at least one fucosylated oligosaccharide; about 10 to about 35 wt %, about 10 to about 30 wt %, or about 10 to about 25 wt %, with respect to the total weight of the prebiotic agent, of at least one sialylated oligosaccharide; and about 10 to about 35 wt %, about 15 to about 30 wt %, or about 15 to about 20 wt %, with respect to the total weight of the prebiotic agent, of at least one N-acetylated oligosaccharide.

The HMOs may be obtained by any suitable method. Suitable methods for synthesising oligosaccharides will be well known to those of skill in the art. For example, processes have been developed for producing oligosaccharides by microbial fermentations, enzymatic processes, chemical syntheses, or combinations of these technologies (see e.g. Zeuner et al., 2019. Molecules, 24 (11), p. 2033).

### Fucosylated Oligosaccharides

In some embodiments, the prebiotic agent comprises at least one fucosylated oligosaccharide.

Non-limiting example(s) of fucosylated oligosaccharide(s) include: 2′-fucosyllactose (2′FL), 3-fucosyllactose (3FL), difucosyllactose (diFL), lacto-N-fucopentaose, such as lacto-N-fucopentaose I (LNFP-I), lacto-N-fucopentaose II (LNFP-II), lacto-N-fucopentaose III (LNFP-III) or lacto-N-fucopentaose V (LNFP-V), lacto-N-fucohexaose, lacto-N-difucohexaose I, lacto-neofucopentaose V (LNnFP-V), lacto-N-difucosylhexaose-I (LNDFH-1), lacto-N-neodifucosylhexaose (LNnDFH), fucosyllacto-N-hexaose, fucosyllacto-N-neohexaose (such as fucosyllacto-N-neohexaose I, fucosyllacto-N-neohexaose II), monofucosyllacto-n-hexaose-III (MFNLH-III), difucosyllacto-N-hexaose I, difuco-lacto-N-neohexaose, difucosyllacto-N-neohexaose I, difucosyllacto-N-neohexaose II, difucosyllacto-N-hexaose-a (DFLNHa), fucosyl-para-Lacto-N-hexaose, tri-fuco-para-Lacto-N-hexaose I, and combinations thereof.

In preferred embodiments, the at least one fucosylated oligosaccharide comprises 2′-fucosyllactose (2′FL), which is typically the most prevalent HMO naturally present in human breast milk.

In some embodiments, the at least one fucosylated oligosaccharide is selected from the group consisting of 2′-fucosyllactose (2′FL), difucosyllactose (diFL) and combinations thereof. In some embodiments, the at least one fucosylated oligosaccharide comprises or consists of 2′-fucosyllactose (2′FL) and difucosyllactose (diFL).

The fucosylated oligosaccharides may be obtained by any suitable method. For example, 2′FL may be produced by biotechnological means using specific fucosyltransferases and/or fucosidases either through the use of enzyme-based fermentation technology (recombinant or natural enzymes) or microbial fermentation technology. In the latter case, microbes may either express their natural enzymes and substrates or may be engineered to produce respective substrates and enzymes. Alternatively, 2′FL may be produced by chemical synthesis from lactose and free fucose. diFL may be synthesized by enzymatic, biotechnological and/or chemical processes.

### Sialylated Oligosaccharides

In some embodiments, the prebiotic agent comprises at least one sialylated oligosaccharide.

Non-limiting examples of sialylated oligosaccharides include: 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof.

In some embodiments, the at least one sialylated oligosaccharide is selected from the group consisting of 3′-sialyllactose (3′-SL), 6′-sialyllactose (6′-SL) and combinations thereof. In some embodiments, the at least one sialylated oligosaccharide comprises or consists of 6′-sialyllactose (6′-SL). In some embodiments, the at least one sialylated oligosaccharide comprises or consists of 6′-sialyllactose (6′-SL) and 3′-sialyllactose (3′-SL).

The sialylated oligosaccharides may be obtained by any suitable method. For example, 3′-sialyllactose (3′-SL), and/or 6′-sialyllactose (6′-SL) may be isolated by chromatographic or filtration technology from a natural source such as animal milks. Alternatively, they may be produced by biotechnological means using specific sialyltransferases or sialidases, neuraminidases, either by an enzyme based fermentation technology (recombinant or natural enzymes), by chemical synthesis or by a microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerisation (DP), from DP=1 onwards. Alternatively, sialyllactoses may be produced by chemical synthesis from lactose and free N′-acetylneuraminic acid (sialic acid). Sialyllactoses are also commercially available for example from Kyowa Hakko Kogyo, Japan, or from GeneChem, Republic of Korea.

If the prebiotic agent comprises 3′-Sialyllactose (3′-SL) and 6′-Sialyllactose (6′-SL), it may be particularly beneficial if said 3′-Sialyllactose (3′-SL) and 6′-Sialyllactose (6′-SL) are comprised in said nutritional composition in a weight ratio between about 10:1 and about 1:10, such as between about 10:1 and about 2:1, between about 8:1 and about 3:1, between about 6:1 and about 3:1, between about 5:1 and about 3:1, between about 5:1 and about 4:1, or between about 1:2 and about 1.5:1.

### N-Acetylated Oligosaccharides

In some embodiments, the prebiotic agent comprises at least one N-acetylated oligosaccharide.

Suitably, the at least one N-acetylated oligosaccharide is selected from the group consisting of N-acetyl-glucosamine, N-acetyl-galactosamines and combinations thereof. Non-limiting examples of N-acetylated oligosaccharide(s) include: LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

In some embodiments, the at least one N-acetylated oligosaccharide is selected from the group consisting of lacto-N-tetraose (LNT), lacto-N-neotetraose (LNnT) and combinations thereof. In some embodiments, the at least one N-acetylated oligosaccharide comprises or consists of lacto-N-tetraose (LNT) and lacto-N-neotetraose (LNnT).

The N-acetylated oligosaccharides may be obtained by any suitable method. For example, LNnT may be synthesised chemically by enzymatic transfer of saccharide units from donor moieties to acceptor moieties using glycosyltransferases. Alternatively, LNnT may be prepared by chemical conversion of Keto-hexoses (e.g. fructose) either free or bound to an oligosaccharide (e.g. lactulose) into N-acetylhexosamine or an N-acetylhexosamine-containing oligosaccharide. LNT may be synthesized by enzymatic, biotechnological and/or chemical processes.

### Cellobiose and Cello-Oligosaccharides (COS)

The prebiotic agent used in the present invention may comprise or consist of cellobiose and/or cello-oligosaccharides (COS).

Cellobiose is a disaccharide with the formula (C₆H₇(OH)₄O)₂O that is derived from the condensation of a pair of β-glucose molecules forming a β(1→4) bond. Cellobiose can be obtained by enzymatic or acidic hydrolysis of cellulose and cellulose-rich materials. Suitably, the cellobiose is in the form of free cellobiose or cello-oligosaccharides.

In some embodiments, the cellobiose is in the form of cello-oligosaccharides (COS). Cello-oligosaccharides may refer to oligomers of β-glucose molecules (e.g. from about 2 to about 6 β-glucose molecules) with 3-1,4-linkages, and may contain mainly cellobiose. The prebiotic agent used in the present invention may comprise or consist of cello-oligosaccharides (COS). Studies have shown that COS has prebiotic potential (see e.g. Zhong, C., et al., 2020. Journal of agricultural and food chemistry, 68 (32), pp. 8557-8567).

### Administration of Prebiotics

The subject may be administered any suitable amounts of prebiotic, in any suitable form and via any suitable route of administration (e.g. in any form and via any route described herein).

For example, suitable doses of human oligosaccharides are described in e.g. EFSA Panel on Dietetic Products, Nutrition and Allergies (NDA), 2015. EFSA Journal, 13 (11), p. 4299; EFSA Panel on Nutrition, Novel Foods and Food Allergens (NDA), 2019. EFSA Journal, 17 (6), p.e05717; EFSA Panel on Nutrition, Novel Foods and Food Allergens (NDA), 2020. EFSA Journal, 18 (5), p.e06097; EFSA Panel on Nutrition, Novel Foods and Food Allergens (NDA), 2022. EFSA Journal, 20 (5), p.e07331; and EFSA Panel on Nutrition, Novel Foods and Food Allergens (NDA), 2019. EFSA Journal, 17 (12), p.e05907.

Suitably, the prebiotic agent is administered to the subject in an amount of at least about 0.5 g/day, at least about 1 g/day, or at least about 2 g/day. Suitably, the prebiotic agent is administered to the subject in an amount of about 10 g/day or less, about 8 g/day or less, or about 5 g/day or less. Suitably, the prebiotic agent is administered to the subject in an amount of from about 0.5 g/day to about 10 g/day, from about 1 g/day to about 8 g/day or from about 2 g/day to about 5 g/day.

Suitably, BMOs are administered to the subject in an amount of at least about 0.5 g/day, at least about 1 g/day, or at least about 2 g/day. Suitably, BMOs are administered to the subject in an amount of about 10 g/day or less, about 8 g/day or less, or about 5 g/day or less. Suitably, BMOs are administered to the subject in an amount of from about 0.5 g/day to about 10 g/day, from about 1 g/day to about 8 g/day or from about 2 g/day to about 5 g/day.

Suitably, HMOs are administered to the subject in an amount of at least about 0.5 g/day, at least about 1 g/day, or at least about 2 g/day. Suitably, HMOs are administered to the subject in an amount of about 10 g/day or less, about 8 g/day or less, or about 5 g/day or less. Suitably, HMOs are administered to the subject in an amount of from about 0.5 g/day to about 10 g/day, from about 1 g/day to about 8 g/day or from about 2 g/day to about 5 g/day.

Suitably, COS is administered to the subject in an amount of at least about 0.5 g/day, at least about 1 g/day, or at least about 2 g/day. Suitably, COS is administered to the subject in an amount of about 10 g/day or less, about 8 g/day or less, or about 5 g/day or less. Suitably, COS is administered to the subject in an amount of from about 0.5 g/day to about 10 g/day, from about 1 g/day to about 8 g/day or from about 2 g/day to about 5 g/day.

### Combination Therapies

The prebiotic agent may be administered in combination with one or more further agents, mixtures, or compositions.

As used herein, a “combination therapy” may refer to a therapy comprising the administration of two or more agents (e.g. a prebiotic agent, one or more vitamins, and/or a probiotic agent), mixtures, or compositions.

The combination may be administered by any suitable route and in any suitable form. Suitably, the combination is administered by oral and/or enteral administration. In preferred embodiments, the combination is administered by oral administration. The combination may be administered separately, simultaneously or sequentially. In preferred embodiments, the combination is administered simultaneously.

In one aspect, the present invention provides a combination of a prebiotic agent, one or more vitamin (e.g. vitamin K1) and one or more probiotic for use in treating and/or preventing vitamin K2 deficiency in a subject.

In another aspect, the present invention provides use of a combination of a prebiotic agent, one or more vitamin (e.g. vitamin K1) and one or more probiotic in the manufacture of a medicament for treating and/or preventing vitamin K2 deficiency in a subject.

In another aspect, the present invention provides a method for treating and/or preventing vitamin K2 deficiency in a subject in need thereof, the method comprising administering to the subject an effective amount of a combination of a prebiotic agent, one or more vitamin (e.g. vitamin K1) and one or more probiotic.

In another aspect, the present invention provides use of a combination of a prebiotic agent, one or more vitamin (e.g. vitamin K1) and one or more probiotic to promote vitamin K2 production in the gut of a subject.

In another aspect, the present invention provides a method for promoting vitamin K2 production in the gut of a subject, the method comprising administering to the subject an effective amount of a combination of a prebiotic agent, one or more vitamin (e.g. vitamin K1) and one or more probiotic.

### Vitamins

The prebiotic agent may be administered in combination with one or more vitamin. The prebiotic agent and one or more vitamin may be administered separately, simultaneously or sequentially. In preferred embodiments, the prebiotic agent and one or more vitamin are administered simultaneously.

Vitamins are organic micronutrients required by the body to carry out a range of normal functions and include vitamin K1, vitamin A, vitamin D, vitamin C, folate, vitamin B3, vitamin B6, vitamin B12, and vitamin E. Suitably, the one or more vitamins may comprise or consist of vitamin K1, vitamin K2, vitamin A, and/or vitamin D. In preferred embodiments, the one or more vitamins comprises or consists of vitamin K1.

In one aspect, the present invention provides a combination of a prebiotic agent and one or more vitamins for use in treating and/or preventing vitamin K2 deficiency in a subject.

In another aspect, the present invention provides use of a combination of a prebiotic agent and one or more vitamins in the manufacture of a medicament for treating and/or preventing vitamin K2 deficiency in a subject.

In another aspect, the present invention provides a method for treating and/or preventing vitamin K2 deficiency in a subject in need thereof, the method comprising administering to the subject an effective amount of a combination of a prebiotic agent and one or more vitamins.

In another aspect, the present invention provides use of a combination of a prebiotic agent and one or more vitamins to promote vitamin K2 production in the gut of a subject.

In another aspect, the present invention provides a method for promoting vitamin K2 production in the gut of a subject, the method comprising administering to the subject an effective amount of a combination of a prebiotic agent and one or more vitamins.

### Vitamin K1

In preferred embodiments, the prebiotic agent may be administered in combination with vitamin K1. The prebiotic agent and vitamin K1 may be administered separately, simultaneously or sequentially. In preferred embodiments, the prebiotic agent and vitamin K1 are administered simultaneously.

In one aspect, the present invention provides a combination of a prebiotic agent and vitamin K1 for use in treating and/or preventing vitamin K2 deficiency in a subject.

In another aspect, the present invention provides use of a combination of a prebiotic agent and vitamin K1 in the manufacture of a medicament for treating and/or preventing vitamin K2 deficiency in a subject.

In another aspect, the present invention provides a method for treating and/or preventing vitamin K2 deficiency in a subject in need thereof, the method comprising administering to the subject an effective amount of a combination of a prebiotic agent and vitamin K1.

In another aspect, the present invention provides use of a combination of a prebiotic agent and vitamin K1 to promote vitamin K2 production in the gut of a subject.

In another aspect, the present invention provides a method for promoting vitamin K2 production in the gut of a subject, the method comprising administering to the subject an effective amount of a combination of a prebiotic agent and vitamin K1.

Vitamin K represents a family of fat-soluble compounds with the common chemical structure of 3-substituted 2-methyl-1,4-napthoquinone. It naturally occurs in food as phylloquinone (vitamin K1) and menaquinones (vitamin K2). Vitamin K1 (phylloquinone) may have the general formula below:

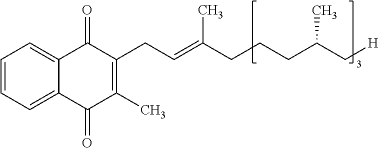

Vitamin K1 has a phytyl side chain and is typically the primary dietary form of vitamin K, and is found in dark green leafy vegetables (e.g. spinach, lettuce and other salad plants) and Brassica.

The subject may be administered any suitable amounts of vitamin K1, in any suitable form and via any suitable route of administration (e.g. in any form and via any route described herein). Suitable doses of vitamin K1 are described in e.g. Kozioł-Kozakowska, A. and Maresz, K., 2022. Children, 9 (1), p. 78 and EFSA Panel on Dietetic Products, Nutrition and Allergies (NDA), 2017. EFSA Journal, 15 (5), p.e04780.

Suitably, vitamin K1 is administered to the subject in an amount of at least about 0.2 μg/kg/day, at least about 0.5 μg/kg/day, or at least about 0.8 μg/kg/day. Suitably, vitamin K1 is administered to the subject in an amount of about 2 μg/kg/day or less, about 1.5 μg/kg/day or less, or about 1.2 μg/kg/day or less. Suitably, vitamin K1 is administered to the subject in an amount of from about 0.2 μg/kg/day to about 2 μg/kg/day, from about 0.5 μg/kg/day to about 1.5 μg/kg/day or from about 0.8 μg/kg/day to about 1.2 μg/kg/day. In some embodiments, vitamin K1 is administered to the subject in an amount of about 1 μg/kg/day

Suitably, vitamin K1 is administered to the subject in an amount of at least about 5 μg/day, at least about 10 μg/day, at least about 15 μg/day, at least about 20 μg/day, at least about 25 μg/day, or at least about 30 μg/day. Suitably, vitamin K1 is administered to the subject in an amount of about 200 μg/day or less, about 100 μg/day or less, about 90 μg/day or less, about 80 μg/day or less, about 70 μg/day or less, or about 60 μg/day or less. Suitably, vitamin K1 is administered to the subject in an amount of from about 5 μg/day to about 200 μg/day, from about 10 μg/day to about 100 μg/day, from about 15 μg/day to about 90 μg/day, from about 20 μg/day to about 80 μg/day, from about 25 μg/day to about 70 μg/day, or from about 30 μg/day to about 60 μg/day.

Suitably, the prebiotic agent is administered to the subject in an amount of from about 0.5 g/day to about 10 g/day and vitamin K1 is administered to the subject in an amount of from about 5 μg/day to about 200 μg/day.

### Vitamin K2

Whereas phylloquinone is a dietary vitamin K found in plants, menaquinones are vitamin K quinones that are both consumed in the diet and produced by the gut microbiota (see e.g. Walther, B., et al., 2013. Advances in nutrition, 4 (4), pp. 463-473). In some embodiments, the prebiotic agent promotes de novo menaquinone-7 production in the subject's gut (e.g. by the gut microbiota). In some embodiments, the prebiotic agent promotes bioconversion from phylloquinone to menaquinone-4 in the subject's gut (e.g. by the gut microbiota).

Vitamin K2 (menaquinone) may have the general formula below:

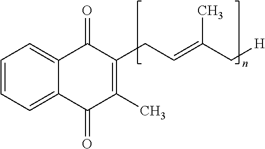

Vitamin K2 consists of various forms, differing in the number (n) of isoprenyl units, wherein n may range from 4 to 13. The various forms are indicated by a suffix (−n), for example, menaquinone-4 (abbreviated MK-4) has four isoprene residues (n=4). MK-4 may be formed via metabolic conversion of phylloquinone during its absorption in the intestinal mucosa and in other organs. Other menaquinones may be produced in the gastrointestinal tract by the gut microbiota.

The prebiotic agent may be administered in combination with vitamin K2. The prebiotic agent and vitamin K2 may be administered separately, simultaneously or sequentially. In preferred embodiments, the prebiotic agent and vitamin K2 are administered simultaneously.

In one aspect, the present invention provides a combination of a prebiotic agent and vitamin K2 for use in treating and/or preventing vitamin K2 deficiency in a subject.

In another aspect, the present invention provides use of a combination of a prebiotic agent and vitamin K2 in the manufacture of a medicament for treating and/or preventing vitamin K2 deficiency in a subject.

In another aspect, the present invention provides a method for treating and/or preventing vitamin K2 deficiency in a subject in need thereof, the method comprising administering to the subject an effective amount of a combination of a prebiotic agent and vitamin K2.

The subject may be administered any suitable amounts of vitamin K2, in any suitable form and via any suitable route of administration (e.g. in any form and via any route described herein). Suitable doses of vitamin K2 are described in e.g. Kozioł-Kozakowska, A. and Maresz, K., 2022. Children, 9 (1), p. 78 and European Food Safety Authority (EFSA), 2008. EFSA Journal, 6(11), p. 822.

Suitably, vitamin K2 is administered to the subject in an amount of at least about 0.5 μg/kg/day, at least about 1 μg/kg/day, or at least about 2 μg/kg/day. Suitably, vitamin K2 is administered to the subject in an amount of about 10 μg/kg/day or less, about 7.5 μg/kg/day or less, or about 5 μg/kg/day or less. Suitably, vitamin K2 is administered to the subject in an amount of from about 0.5 g/kg/day to about 10 μg/kg/day, from about 1 μg/kg/day to about 7.5 μg/kg/day or from about 2 μg/kg/day to about 5 μg/kg/day.

Suitably, vitamin K2 is administered to the subject in an amount of at least about 5 μg/day, at least about 10 μg/day, at least about 15 μg/day, at least about 20 μg/day, at least about 25 μg/day, or at least about 30 μg/day. Suitably, vitamin K2 is administered to the subject in an amount of about 200 μg/day or less, about 100 μg/day or less, about 90 μg/day or less, about 80 μg/day or less, about 70 μg/day or less, or about 60 μg/day or less. Suitably, vitamin K2 is administered to the subject in an amount of from about 5 μg/day to about 200 μg/day, from about 10 μg/day to about 100 μg/day, from about 15 μg/day to about 90 μg/day, from about 20 μg/day to about 80 μg/day, from about 25 μg/day to about 70 μg/day, or from about 30 μg/day to about 60 μg/day.

### Vitamin A

Vitamin A comprises a family of molecules containing a 20 carbon structure with a methyl substituted cyclohexenyl ring (beta-ionone ring) and a tetraene side chain with a hydroxyl group (retinol), aldehyde group (retinal), carboxylic acid group (retinoic acid), or ester group (retinyl ester) at carbon-15. The term vitamin A may also include provitamin A carotenoids that are dietary precursors of retinol. Of the many carotenoids in nature, several have provitamin A nutritional activity, including α-carotene, β-carotene, and β-cryptoxanthin.

The amount of vitamin A may be referred to in retinol equivalents (RE) or retinol activity equivalents (RAE). For dietary provitamin A carotenoids β-carotene, α-carotene, and β-cryptoxanthin, REs have been set at 6, 12, and 12 μg, respectively. Using μg RAE, the vitamin A activity of provitamin A carotenoids is half the vitamin A activity assumed when using μg RE. For dietary provitamin A carotenoids β-carotene, α-carotene, and β-cryptoxanthin, RAEs have been set at 12, 24, and 24 μg, respectively. (See e.g. Dietary Reference Intakes for Vitamin A, Vitamin K, Arsenic, Boron, Chromium, Copper, Iodine, Iron, Manganese, Molybdenum, Nickel, Silicon, Vanadium, and Zinc. Washington (DC): National Academies Press (US); 2001. 4, Vitamin A).

The subject may be administered any suitable amounts of vitamin A, in any suitable form and via any suitable route of administration (e.g. in any form and via any route described herein). Suitable doses of vitamin A are described in e.g. Ross, A. C. and Moran, N. E., 2020. Current Developments in Nutrition, 4 (10), p.nzaa096 and EFSA Panel on Dietetic Products, Nutrition, and Allergies (NDA), 2015. EFSA Journal, 13 (3), p. 4028.

Suitably, vitamin A is administered to the subject in an amount of at least about 100 μg RE/day, at least about 200 μg RE/day, or at least about 300 μg RE/day. Suitably, vitamin A is administered to the subject in an amount of about 1000 μg RE/day or less, about 800 μg RE/day or less, about 600 μg RE/day or less, or about 400 μg RE/day or less. Suitably, vitamin A is administered to the subject in an amount of from about 100 μg RE/day to about 1000 μg RE/day, from about 200 μg RE/day to about 800 μg RE/day, from about 300 μg RE/day to about 600 μg RE/day, or from about 300 μg RE/day to about 400 μg RE/day.

Suitably, vitamin A is administered to the subject in an amount of at least about 100 μg RE/day, at least about 200 μg RAE/day, or at least about 300 μg RAE/day. Suitably, vitamin A is administered to the subject in an amount of about 1000 μg RAE/day or less, about 800 μg RAE/day or less, about 600 μg RAE/day or less, or about 400 μg RAE/day or less. Suitably, vitamin A is administered to the subject in an amount of from about 100 μg RAE/day to about 1000 μg RAE/day, from about 200 μg RAE/day to about 800 μg RAE/day, from about 300 μg RAE/day to about 600 μg RAE/day, or from about 300 μg RAE/day to about 400 μg RAE/day.

### Vitamin D

Vitamin D is a group of fat-soluble secosteroids including vitamin D2 (ergocalciferol) and vitamin D3 (cholecalciferol). Calcitriol (also known as 1,25-dihydroxyvitamin D) is the active form of vitamin D. Suitably, the vitamin D comprises or consists of vitamin D2 and vitamin D3. Suitably, the vitamin D comprises or consists of calcitriol.

The subject may be administered any suitable amounts of vitamin D, in any suitable form and via any suitable route of administration (e.g. in any form and via any route described herein). Suitable doses of vitamin D are described in e.g. Greer, F. R., 2004. The American journal of clinical nutrition, 80 (6), pp. 1759S-1762S and EFSA Panel on Dietetic Products, Nutrition and Allergies (NDA), 2016. EFSA Journal, 14 (10), p.e04547.

Suitably, vitamin D is administered to the subject in an amount of at least about 2.5 μg/day, at least about 5 μg/day, at least about 10 μg/day, or at least about 15 μg/day. Suitably, vitamin D is administered to the subject in an amount of about 100 μg/day or less, about 75 μg/day or less, or about 50 μg/day or less. Suitably, vitamin D is administered to the subject in an amount of from about 2.5 μg/day to about 100 μg/day, from about 5 μg/day to about 100 μg/day, from about 10 μg/day to about 75 μg/day or from about 15 μg/day to about 50 μg/day. Suitably, vitamin D is administered to the subject in an amount of about 15 μg/day.

### Vitamin Mixture

The prebiotic agent may be administered in combination with a vitamin mixture. The prebiotic agent and vitamin mixture may be administered separately, simultaneously or sequentially. In preferred embodiments, the prebiotic agent and vitamin mixture are administered simultaneously.

In one aspect, the present invention provides a combination of a prebiotic agent and a vitamin mixture for use in treating and/or preventing vitamin K2 deficiency in a subject.

In another aspect, the present invention provides use of a combination of a prebiotic agent and a vitamin mixture in the manufacture of a medicament for treating and/or preventing vitamin K2 deficiency in a subject.

In another aspect, the present invention provides a method for treating and/or preventing vitamin K2 deficiency in a subject in need thereof, the method comprising administering to the subject an effective amount of a combination of a prebiotic agent and a vitamin mixture.

In another aspect, the present invention provides use of a combination of a prebiotic agent and a vitamin mixture to promote vitamin K2 production in the gut of a subject.

As used herein, a “vitamin mixture” may refer to a mixture of two or more vitamins. Any suitable vitamin mixture may be used.

In some embodiments, the vitamin mixture comprises or consists of vitamin K1, vitamin A, and vitamin D. Suitably, vitamin K1 is administered to the subject in an amount of from about 5 μg/day to about 200 μg/day, vitamin A is administered to the subject in an amount of from about 100 μg RE/day to about 1000 μg RE/day, and vitamin D is administered to the subject in an amount of from about 2.5 μg/day to about 100 μg/day.

In other embodiments, the vitamin mixture comprises or consists of vitamin K2, vitamin A, and vitamin D. Suitably, vitamin K2 is administered to the subject in an amount of from about 5 μg/day to about 200 μg/day, vitamin A is administered to the subject in an amount of from about 100 μg RE/day to about 1000 μg RE/day, and vitamin D is administered to the subject in an amount of from about 2.5 μg/day to about 100 μg/day.

In further embodiments, the present invention provides use of a combination of a prebiotic agent and vitamin K2 for treating and/or preventing vitamin K2 deficiency in a subject.

In further embodiments, the present invention provides use of a combination of a prebiotic agent and vitamin A for treating and/or preventing vitamin K2 deficiency in a subject.

In further embodiments, the present invention provides use of a combination of a prebiotic agent and vitamin D for treating and/or preventing vitamin K2 deficiency in a subject.

In further embodiments, the present invention provides use of a combination of a prebiotic agent and vitamin K2, vitamin A, vitamin D, or any combination thereof for treating and/or preventing vitamin K2 deficiency in a subject.

### Probiotics

The prebiotic agent may be administered in combination with one or more probiotic. The prebiotic agent and one or more probiotic may be administered separately, simultaneously or sequentially. In preferred embodiments, the prebiotic agent and one or more probiotic are administered simultaneously.

As used herein, the term “probiotic” may refer to a component that contains a sufficient number of viable microorganisms to alter the gut microbiota of the subject (see e.g. Hill, C., et al., 2014. Nature reviews Gastroenterology & hepatology, 11 (8), p. 506). Suitably, the probiotic comprises a commercially available probiotic strain and/or a strain which has been shown to have health benefits (See e.g. Fijan, S., 2014. International journal of environmental research and public health, 11 (5), pp. 4745-4767). Exemplary probiotic microorganisms may include Bifidobacterium, Lactobacillus, Limosilactobacillus, Lacticaseibacillus, Saccharomyces, Enterococcus, Streptococcus, Pediococcus, Leuconostoc, Bacillus, and Escherichia coli.

In one aspect, the present invention provides a combination of a prebiotic agent and one or more probiotic for use in treating and/or preventing vitamin K2 deficiency in a subject.

In another aspect, the present invention provides use of a combination of a prebiotic agent and one or more probiotic in the manufacture of a medicament for treating and/or preventing vitamin K2 deficiency in a subject.

In another aspect, the present invention provides a method for treating and/or preventing vitamin K2 deficiency in a subject in need thereof, the method comprising administering to the subject an effective amount of a combination of a prebiotic agent and one or more probiotic.

In another aspect, the present invention provides use of a combination of a prebiotic agent and one or more probiotic to promote vitamin K2 production in the gut of a subject.

In another aspect, the present invention provides a method for promoting vitamin K2 production in the gut of a subject, the method comprising administering to the subject an effective amount of a combination of a prebiotic agent and one or more probiotic.

The one or more probiotic may increase vitamin K2 production. The present inventors have surprisingly found that probiotics may boost endogenous production of vitamin K2, through the production of precursors.

In some embodiments, the one or more probiotic comprises or consists of Lacticaseibacillus, Bifidobacterium, Lactobacillus, and/or Limosilactobacillus. In some embodiments, the one or more probiotic comprises or consists of Lactobacillus rhamnosus, Bifidobacterium longum and/or Bifidobacterium animalis.

Lactobacillus rhamnosus

In some embodiments, the one or more probiotic comprises or consists of Lactobacillus rhamnosus.

Lacticaseibacillus rhamnosus (also known as Lactobacillus rhamnosus) is a short Gram-positive homofermentative facultative anaerobic non-spore-forming rod that often appears in chains. Lactobacillus rhamnosus GG (LGG) is one of the most widely used probiotic strains. Various health effects are well documented (see e.g. Segers, M. E. and Lebeer, S., 2014. Microbial cell factories, 13 (1), pp. 1-16). L. rhamnosus may boost endogenous production of vitamin K2, through the production of precursors.

In some embodiments, the one or more probiotic comprises or consists of Lactobacillus rhamnosus LPR.

Bifidobacterium longum

In some embodiments, the one or more probiotic comprises or consists of Bifidobacterium longum.

Bifidobacterium longum is a bacterium present in the human gastrointestinal tract. In 2002, three previously distinct species of Bifidobacterium, B. infantis, B. longum, and B. suis, were unified into a single species named B. longum with the biotypes infantis, longum, and suis, respectively (Sakata, S., et al., 2002. International journal of systematic and evolutionary microbiology, 52 (6), pp. 1945-1951).

In some embodiments, the one or more probiotic comprises or consists of Bifidobacterium longum ssp. infantis (also known as Bifidobacterium infantis), Bifidobacterium longum ssp. suis (also known as Bifidobacterium suis), and/or Bifidobacterium longum ssp. longum (also known as Bifidobacterium longum).

In some embodiments, the one or more probiotic comprises or consists of Bifidobacterium infantis. In some embodiments, the one or more probiotic comprises or consists of Bifidobacterium infantis LMG 11588 or a derivative thereof (e.g. R0033 which has been communicated to the US Food and Drug Administration as generally recognized as safe (GRAS), see e.g. Duboux, S., et al., 2022. Microorganisms, 10 (2), p. 203). B. infantis may boost endogenous production of vitamin K2, through the production of precursors.

Bifidobacterium animalis

In some embodiments, the one or more probiotic comprises or consists of Bifidobacterium animalis.

Bifidobacterium animalis is a bacterium of the Bifidobacterium genus which can be found in the large intestines of most mammals, including humans. Bifidobacterium animalis and Bifidobacterium lactis were previously described as two distinct species. Presently, both are considered B. animalis with the subspecies Bifidobacterium animalis subsp. animalis and Bifidobacterium animalis subsp. lactis (see Masco, L., et al., 2004. International Journal of Systematic and Evolutionary Microbiology, 54 (4), pp. 1137-1143).

In some embodiments, the one or more probiotic comprises or consists of Bifidobacterium animalis ssp. lactis (also known as Bifidobacterium lactis). For example, the bacterium, Bifidobacterium lactis HN019, has been studied for a variety of traits important to its ability to function as a probiotic (see e.g. Sanders, M. E., 2006. Journal of clinical gastroenterology, 40 (9), pp. 776-783).

### Administration of Probiotics

The subject may be administered any suitable amounts of probiotics, in any suitable form and via any suitable route of administration (e.g. in any form and via any route described herein).

Suitably, the one or more probiotic is administered to the subject in a total amount of at least about 10⁵ cfu/day, at least about 10⁶ cfu/day, at least about 10⁷ cfu/day, at least about 10⁸ cfu/day, at least about 10⁹ cfu/day, or at least about 10¹⁰ cfu/day. Suitably, the one or more probiotic is administered to the subject in a total amount of about 10¹² cfu/day or less, about 10¹¹ cfu/day or less, or about 10¹⁰ cfu/day or less. Suitably, the one or more probiotic is administered to the subject in a total amount of from about 10⁶ cfu/day to about 10¹² cfu/day, from about 10⁷ cfu/day to about 10¹¹ cfu/day or from about 10⁸ cfu/day to about 10¹⁰ cfu/day.

Suitably, Lactobacillus rhamnosus is administered to the subject in an amount of at least about 10⁵ cfu/day, at least about 10⁶ cfu/day, at least about 10⁷ cfu/day, at least about 10⁸ cfu/day, at least about 10⁹ cfu/day, or at least about 10¹⁰ cfu/day. Suitably, Lactobacillus rhamnosus is administered to the subject in an amount of about 10¹² cfu/day or less, about 10¹¹ cfu/day or less, or about 10¹⁰ cfu/day or less. Suitably, Lactobacillus rhamnosus is administered to the subject in an amount of from about 10⁶ cfu/day to about 10¹² cfu/day, from about 10⁷ cfu/day to about 10¹¹ cfu/day or from about 10⁸ cfu/day to about 10¹⁰ cfu/day.

Suitably, Bifidobacterium longum (e.g. B. infantis) is administered to the subject in an amount of at least about 10⁵ cfu/day, at least about 10⁶ cfu/day, at least about 10⁷ cfu/day, at least about 10⁸ cfu/day, at least about 10⁹ cfu/day, or at least about 10¹⁰ cfu/day. Suitably, Bifidobacterium longum (e.g. B. infantis) is administered to the subject in an amount of about 10¹² cfu/day or less, about 10¹¹ cfu/day or less, or about 10¹⁰ cfu/day or less. Suitably, Bifidobacterium longum (e.g. B. infantis) is administered to the subject in an amount of from about 10⁶ cfu/day to about 10¹² cfu/day, from about 10⁷ cfu/day to about 10¹¹ cfu/day or from about 10⁸ cfu/day to about 10¹⁰ cfu/day.

Suitably, Bifidobacterium animalis (e.g. B. lactis) is administered to the subject in an amount of at least about 10⁵ cfu/day, at least about 10⁶ cfu/day, at least about 10⁷ cfu/day, at least about 10⁸ cfu/day, at least about 10⁹ cfu/day, or at least about 10¹⁰ cfu/day. Suitably, Bifidobacterium animalis (e.g. B. lactis) is administered to the subject in an amount of about 10¹² cfu/day or less, about 10¹¹ cfu/day or less, or about 10¹⁰ cfu/day or less. Suitably, Bifidobacterium animalis (e.g. B. lactis) is administered to the subject in an amount of from about 10⁶ cfu/day to about 10¹² cfu/day, from about 10⁷ cfu/day to about 10¹¹ cfu/day or from about 10⁸ cfu/day to about 10¹⁰ cfu/day.

Suitably, the prebiotic agent is administered to the subject in an amount of from about 0.5 g/day to about 10 g/day and one or more probiotic is administered to the subject in a total amount of from about 10⁶ cfu/day to about 10¹² cfu/day.

Suitably, the prebiotic agent is administered to the subject in an amount of from about 0.5 g/day to about 10 g/day, vitamin K1 is administered to the subject in an amount of from about 5 μg/day to about 200 μg/day, and one or more probiotic is administered to the subject in a total amount of from about 10⁶ cfu/day to about 10¹² cfu/day.

### Compositions

Suitably, the prebiotic agent (or combination therewith) is in the form of a composition. The composition may comprise the combination in any therapeutically effective amount.

The composition can be any type of composition in which the combination can be incorporated, such as a composition in the form of a food or beverage product, an animal feed product, a nutritional supplement for human or animal, or a pharmaceutical composition. The composition may be in solid (e.g. powder), liquid or semi-liquid form. The combination may be in the form of a food composition, a pet food composition, a beverage, a nutritional formula, a nutritional supplement, or a nutraceutical.

Food and beverage products include all products intended to be consumed orally by human beings, for the purpose of providing nutrition and/or pleasure. It can for example be a nutritional composition, such as for young children. Examples of food and beverage products include dairy products such as milk products or yogurts, soups, sauces, sweet and savoury snacks, powdered drinks and cereal products.

In some embodiments, the prebiotic agent (or combination therewith) is in the form of a nutritional composition, a medical food product for clinical nutrition, or a supplement.

In some embodiments, the prebiotic agent (or combination therewith) is in the form of a nutritional composition. As used herein, a “nutritional composition” may mean a composition which nourishes a subject. This nutritional composition is usually to be taken orally or intravenously, and typically includes a lipid or fat source and a protein source.

In some embodiments, the prebiotic agent (or combination therewith) is in the form of a medical food product for clinical nutrition. As used herein, a “medical food product for clinical nutrition” may also be known as a “Food for Special Medical Purposes (FSMP)” and refer to specialised foods designed to help meet the nutritional or dietary needs of subjects living with a disease, disorder or medical condition who are temporarily or permanently unable to achieve an adequate nutritional intake from normal foods or through modification of the normal diet.

In some embodiments, the prebiotic agent (or combination therewith) is in the form of an infant formula. In this case, said infant formula can be a preterm infant formula, a human milk fortifier, a starter infant formula, a follow-on formula, a baby-food formula, an infant cereal formula, or a growing-up milk.

In some embodiments, the prebiotic agent (or combination therewith) is in the form of a growing-up milk. The term “growing-up milk” (or GUM) as used herein refers to a milk formula product given from one year onwards. It is generally a diary-based beverage adapted for the specific nutritional needs of young children (e.g. children aged from about 1 to about 3). Growing-up milk may also be known as “young-child formula” or “toddlers' milk”.

In some embodiments, the prebiotic agent (or combination therewith) is in the form of a milk formula. The term “milk formula” as used herein may refer to a foodstuff intended for e.g. childhood nutrition, which may provide the sole source or supplemental nutrition for children e.g. aged about 3 years or older. In some embodiments, the milk formula is a grow milk.

In some embodiments, the composition is in a powder form and reconstituted in an aqueous medium (e.g. water) prior to administration. In other embodiments, the composition is in a liquid form ready for administration (e.g. a ready-to-feed formula).

In another embodiment, the prebiotic agent (or combination therewith) is in the form of a supplement. As used herein, a “supplement” or “dietary supplement” may be used to complement the nutrition of a subject (it is typically used as such but it might also be added to any kind of compositions intended to be ingested by the subject). When the composition is a supplement, it can be provided in the form of unit doses. Supplements are typically present in the form of a liquid, a gel, a powder or a tablet or capsule. Powder supplements typically encompass supplements to be dissolved in water or milk, or to be sprinkled on food or in a beverage. Such supplements are intended to provide additional nutrients and/or a health benefit to the subject consuming it. A supplement can be used for providing nutrients and/or a health benefit to human beings, as well as to animals.

In another embodiment, the prebiotic agent (or combination therewith) is in the form of a fortifier. The fortifier can be an infant formula fortifier or growing-up milk fortifier.

In another embodiment, the prebiotic agent (or combination therewith) is in the form of a pharmaceutical product. Pharmaceutical products include for example drops, syrups, powder, tablet or capsule products intended to treat of prevent an adverse medical condition in a subject in need thereof.

The prebiotic agent (or combination therewith) can also be in the form of an animal food product or a nutritional supplement for animals. Preferably, the animal is a mammal. Examples of animals include primates (e.g., humans), cows, sheep, goats, horses, dogs, cats, rabbits, rats, mice, fish, birds and the like.

### Prebiotics

A nutritional composition according to the invention may contain any suitable amounts of the prebiotic agent.

Suitably, the nutritional composition comprises the prebiotic agent in a total amount of at least about 0.5 wt %, at least about 1 wt %, at least about 2 wt %, at least about 3 wt %, at least about 4 wt %, or at least about 5 wt % on a dry weight basis. Suitably, the nutritional composition comprises the prebiotic agent in a total amount of about 10 wt % or less, about 8 wt % or less or about 5 wt % or less, on a dry weight basis. Suitably, the nutritional composition comprises the prebiotic agent in a total amount of from about 0.5 wt % to about 10 wt %, about 1 wt % to about 8 wt %, or about 2 wt % to about 5 wt %, on a dry weight basis.

Suitably, the prebiotic agent comprises BMOs in an amount of from about 80 wt % to about 100 wt %, with respect to the total weight of the prebiotic agent. Suitably, the nutritional composition comprises BMOs in a total amount of at least about 0.5 wt %, at least about 1 wt %, at least about 2 wt %, at least about 3 wt %, at least about 4 wt %, or at least about 5 wt % on a dry weight basis. Suitably, the nutritional composition comprises BMOs in a total amount of about 10 wt % or less, about 8 wt % or less or about 5 wt % or less, on a dry weight basis. Suitably, the nutritional composition comprises BMOs in a total amount of from about 0.5 wt % to about 10 wt %, about 1 wt % to about 8 wt %, or about 2 wt % to about 5 wt %, on a dry weight basis.

Suitably, the nutritional composition comprises HMOs in a total amount of at least about 0.5 wt %, at least about 1 wt %, at least about 2 wt %, at least about 3 wt %, at least about 4 wt %, or at least about 5 wt % on a dry weight basis. Suitably, the nutritional composition comprises HMOs in a total amount of about 10 wt % or less, about 8 wt % or less or about 5 wt % or less, on a dry weight basis. Suitably, the nutritional composition comprises HMOs in a total amount of from about 0.5 wt % to about 10 wt %, about 1 wt % to about 8 wt %, or about 2 wt % to about 5 wt %, on a dry weight basis.

Suitably, the nutritional composition comprises at least one fucosylated oligosaccharide in an amount of about 0.05 wt % to about 3 wt %, about 0.1 wt % to about 2 wt %, or about 0.2 wt % to about 1.5 wt %, on a dry weight basis. Suitably, the nutritional composition comprises at least one sialylated oligosaccharide in an amount of about 0.01 wt % to about 2 wt %, about 0.05 wt % to about 1.5 wt %, or about 0.07 wt % to about 1 wt %, on a dry weight basis. Suitably, the nutritional composition comprises at least one N-acetylated oligosaccharide in an amount of about 0.01 wt % to about 1 wt %, about 0.03 wt % to about 0.6 wt %, or about 0.05 wt % to about 0.5 wt %, on a dry weight basis.

Suitably, the nutritional composition comprises 2′FL in a total amount of from about 0.05 g/L to about 2 g/L, from about 0.1 g/L to about 1 g/L, from about 0.15 g/L to about 0.8 g/L, from about 0.2 g/L to about 0.7 g/L, or from about 0.25 g/L to about 0.6 g/L (the concentration may refer to the concentration after the composition has been reconstituted e.g. with water).

Suitably, the nutritional composition comprises fucosylated oligosaccharide(s) (e.g. 2′FL and/or diFL) in a total amount of from about 0.1 g/L to about 4 g/L, from about 0.1 g/L to about 3.5 g/L, from about 0.15 g/L to about 3 g/L, from about 0.2 g/L to about 2.5 g/L, from about 0.3 g/L to about 2 g/L, from about 0.4 g/L to about 2 g/L, or from about 0.5 g/L to about 2 g/L (the concentration may refer to the concentration after the composition has been reconstituted e.g. with water). In a particular embodiment, the nutritional composition comprises from about 0.2 g/L to about 1.8 g/L of total fucosylated oligosaccharide(s).

Suitably, the nutritional composition comprises sialylated oligosaccharide(s) (e.g. 3′-sialyllactose (3′-SL) and/or 6′-sialyllactose (6′-SL) in a total amount of from about 0.05 g/L to about 0.75 g/L, from about 0.05 g/L to about 0.5 g/L, from about 0.1 g/L to about 0.3 g/L, or from about 0.1 g/L to about 0.4 g/L (the concentration may refer to the concentration after the composition has been reconstituted e.g. with water). In a particular embodiment, the nutritional composition comprises from about 0.12 g/L to about 0.4 g/L of total sialylated oligosaccharide(s).

Suitably, the nutritional composition comprises N-acetylated oligosaccharide(s) (e.g. LNT and/or LNnT) in a total amount of from about 0.05 g/L to about 0.5 g/L, from about 0.1 g/L to about 0.5 g/L, from about 0.2 g/L to about 0.4 g/L, or about 0.3 g/L (the concentration may refer to the concentration after the composition has been reconstituted e.g. with water).

Suitably, the nutritional composition comprises:
at least one sialylated oligosaccharide in in a total amount of from about 0.05 g/L to about 0.75 g/L, from about 0.05 g/L to about 0.5 g/L, from about 0.1 g/L to about 0.3 g/L, or from about 0.1 g/L to about 0.4 mg/L;
at least one fucosylated oligosaccharide in a total amount of from about 0.1 g/L to about 4 g/L, from about 0.1 g/L to about 3.5 g/L, from about 0.15 to about 3 g/L, from about 0.2 g/L to about 2.5 g/L, from about 0.3 g/L to about 2 g/L, from about 0.4 g/L to about 2 g/L, or from about 0.5 g/L to about 2 g/L; and/or
at least one N-acetylated oligosaccharide in a total amount of from about 0.05 g/L to about 0.5 g/L, from about 0.1 g/L to about 0.5 g/L, or from about 0.2 g/L to about 0.4 g/L.

Suitably, the nutritional composition comprises COS in an amount of at least about 0.5 wt %, at least about 1 wt %, at least about 2 wt %, at least about 3 wt %, at least about 4 wt %, or at least about 5 wt % on a dry weight basis. Suitably, the nutritional composition comprises COS in an amount of about 10 wt % or less, about 8 wt % or less or about 5 wt % or less, on a dry weight basis. Suitably, the nutritional composition comprises COS in an amount of from about 0.5 wt % to about 10 wt %, about 1 wt % to about 8 wt %, or about 2 wt % to about 5 wt %, on a dry weight basis.

### Vitamins

A nutritional composition according to the invention may contain any suitable amounts of vitamins.

Suitably, the nutritional composition comprises vitamin K1 in an amount of at least about 5 μg/100 g, at least about 10 μg/100 g, at least about 15 μg/100 g, at least about 20 μg/100 g, at least about 25 μg/100 g, or at least about 30 μg/100 g on a dry weight basis. Suitably, the nutritional composition comprises vitamin K1 in an amount of about 2100 μg/100 g or less, about 100 μg/100 g or less, about 90 μg/100 g or less, about 80 μg/100 g or less, about 70 μg/100 g or less, or about 60 μg/100 g or less, on a dry weight basis. Suitably, the nutritional composition comprises vitamin K1 in an amount of from about 5 μg/100 g to about 200 μg/100 g, from about 10 μg/100 g to about 100 μg/100 g, from about 15 μg/100 g to about 90 μg/100 g, from about 20 μg/100 g to about 80 μg/100 g, from about 25 μg/100 g to about 70 μg/100 g, or from about 30 μg/100 g to about 60 μg/100 g.

Suitably, the nutritional composition comprises vitamin K2 in an amount of at least about 5 μg/100 g, at least about 10 μg/100 g, at least about 15 μg/100 g, at least about 20 μg/100 g, at least about 25 μg/100 g, or at least about 30 μg/100 g on a dry weight basis. Suitably, the nutritional composition comprises vitamin K2 in an amount of about 2100 μg/100 g or less, about 100 μg/100 g or less, about 90 μg/100 g or less, about 80 μg/100 g or less, about 70 μg/100 g or less, or about 60 μg/100 g or less, on a dry weight basis. Suitably, the nutritional composition comprises vitamin K2 in an amount of from about 5 μg/100 g to about 200 μg/100 g, from about 10 μg/100 g to about 100 μg/100 g, from about 15 μg/100 g to about 90 μg/100 g, from about 20 μg/100 g to about 80 μg/100 g, from about 25 μg/100 g to about 70 μg/100 g, or from about 30 μg/100 g to about 60 μg/100 g.

Suitably, the nutritional composition comprises vitamin A in an amount of at least about 100 μg RE/100 g, at least about 200 μg RE/100 g, or at least about 300 μg RE/100 g, on a dry weight basis. Suitably, the nutritional composition comprises vitamin A in an amount of about 1000 μg RE/100 g or less, about 800 μg RE/100 g or less, about 600 μg RE/100 g or less, or about 400 μg RE/100 g or less, on a dry weight basis. Suitably, the nutritional composition comprises vitamin A in an amount of from about 100 μg RE/100 g to about 1000 μg RE/100 g, from about 200 μg RE/100 g to about 800 μg RE/100 g, from about 300 μg RE/100 g to about 600 μg RE/100 g, or from about 300 μg RE/100 g to about 400 μg RE/100 g, on a dry weight basis.

Suitably, the nutritional composition comprises vitamin A in an amount of at least about 100 μg RAE/100 g, at least about 200 μg RAE/100 g, or at least about 300 μg RAE/100 g, on a dry weight basis. Suitably, the nutritional composition comprises vitamin A in an amount of about 1000 μg RAE/100 g or less, about 800 μg RAE/100 g or less, about 600 μg RAE/100 g or less, or about 400 μg RAE/100 g or less, on a dry weight basis. Suitably, the nutritional composition comprises vitamin A in an amount of from about 100 μg RAE/100 g to about 1000 μg RAE/100 g, from about 200 μg RAE/100 g to about 800 μg RAE/100 g, from about 300 μg RAE/100 g to about 600 μg RAE/100 g, or from about 300 μg RAE/100 g to about 400 μg RAE/100 g, on a dry weight basis.

Suitably, the nutritional composition comprises vitamin D in an amount of at least about 5 μg/100 g, at least about 10 μg/100 g, or at least about 15 μg/100 g, on a dry weight basis. Suitably, the nutritional composition comprises vitamin D in an amount of about 100 μg/100 g or less, about 75 μg/100 g or less, or about 50 μg/100 g or less, on a dry weight basis. Suitably, the nutritional composition comprises vitamin D in an amount of from about 5 μg/100 g to about 100 μg/100 g, from about 10 μg/100 g to about 75 μg/100 g or from about 15 μg/100 g to about 50 μg/100 g, on a dry weight basis.

### Probiotics

A nutritional composition according to the invention may contain any suitable amounts of probiotic.

Suitably, the nutritional composition comprises the one or more probiotic in a total amount of at least about 10⁵ cfu/100 g, at least about 10⁶ cfu/100 g, at least about 10⁷ cfu/100 g, or at least about 10⁸ cfu/100 g, at least about 10⁹ cfu/100 g, or at least about 10¹⁰ cfu/100 g on a dry weight basis. Suitably, the nutritional composition comprises the one or more probiotic in a total amount of about 10¹² cfu/100 g or less, about 10¹¹ cfu/100 g or less, about 10¹⁰ cfu/100 g or less, on a dry weight basis. Suitably, the nutritional composition comprises the one or more probiotic in a total amount of about 10⁶ cfu/100 g to about 10¹² cfu/100 g, about 10⁷ cfu/100 g to about 10¹¹ cfu/100 g, or about 10⁸ cfu/100 g to about 10¹⁰ cfu/100 g, on a dry weight basis.

Suitably, the nutritional composition comprises Bifidobacterium longum (e.g. B. infantis) in an amount of at least about 10⁵ cfu/100 g, at least about 10⁶ cfu/100 g, at least about 10⁷ cfu/100 g, or at least about 10⁸ cfu/100 g, at least about 10⁹ cfu/100 g, or at least about 10¹⁰ cfu/100 g on a dry weight basis. Suitably, the nutritional composition comprises Bifidobacterium longum (e.g. B. infantis) in an amount of about 10¹² cfu/100 g or less, about 10¹¹ cfu/100 g or less, about 10¹⁰ cfu/100 g or less, on a dry weight basis. Suitably, the nutritional composition comprises Bifidobacterium longum (e.g. B. infantis) in an amount of about 10⁶ cfu/100 g to about 10¹² cfu/100 g, about 10⁷ cfu/100 g to about 10¹¹ cfu/100 g, or about 10⁸ cfu/100 g to about 10¹⁰ cfu/100 g, on a dry weight basis.

Suitably, the nutritional composition comprises Bifidobacterium animalis (e.g. B. lactis) in an amount of at least about 10⁵ cfu/100 g, at least about 10⁶ cfu/100 g, at least about 10⁷ cfu/100 g, or at least about 10⁸ cfu/100 g, at least about 10⁹ cfu/100 g, or at least about 10¹⁰ cfu/100 g on a dry weight basis. Suitably, the nutritional composition comprises Bifidobacterium animalis (e.g. B. lactis) in an amount of about 10¹² cfu/100 g or less, about 10¹¹ cfu/100 g or less, about 10¹⁰ cfu/100 g or less, on a dry weight basis. Suitably, the nutritional composition comprises Bifidobacterium animalis (e.g. B. lactis) in an amount of about 10⁶ cfu/100 g to about 10¹² cfu/100 g, about 10⁷ cfu/100 g to about 10¹¹ cfu/100 g, or about 10⁸ cfu/100 g to about 10¹⁰ cfu/100 g, on a dry weight basis.

Suitably, the nutritional composition comprises Lactobacillus rhamnosus in an amount of at least about 10⁵ cfu/100 g, at least about 10⁶ cfu/100 g, at least about 10⁷ cfu/100 g, or at least about 10⁸ cfu/100 g, at least about 10⁹ cfu/100 g, or at least about 10¹⁰ cfu/100 g on a dry weight basis. Suitably, the nutritional composition comprises Lactobacillus rhamnosus in an amount of about 10¹² cfu/100 g or less, about 10¹¹ cfu/100 g or less, about 10¹⁰ cfu/100 g or less, on a dry weight basis. Suitably, the nutritional composition comprises Lactobacillus rhamnosus in an amount of about 10⁶ cfu/100 g to about 10¹² cfu/100 g, about 10⁷ cfu/100 g to about 1011 cfu/100 g, or about 10⁸ cfu/100 g to about 10¹⁰ cfu/100 g, on a dry weight basis.

### Other Components

A nutritional composition of the invention may contain a protein source, a carbohydrate source and a lipid source. In some embodiments however, especially if a nutritional composition of the invention is a supplement or a fortifier, there may be only lipids (or a lipid source).

The nutritional composition of the invention may comprise about 100 kcal/100 g to about 1000 kcal/100 g, about 200 kcal/100 g to about 800 kcal/100 g, or about 400 kcal/100 g to about 600 kcal/100 g, on a dry weight basis.

### Protein

A nutritional composition according to the invention may contain a protein source. The protein may be in an amount of from about 1 g to about 4 g per 100 kcal, or about 1.5 g to about 3 g per 100 kcal.

Protein sources based on, for example, whey, casein and mixtures thereof may be used as well as plant based protein sources, for example, based on soy. As far as whey proteins are concerned, the protein source may be based on acid whey or sweet whey or mixtures thereof and may include alpha-lactalbumin and beta-lactoglobulin in any desired proportions. In some embodiments the protein source is whey predominant (i.e. more than 50% of proteins are coming from whey proteins, such as 60%> or 70%>). The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10%> by weight of lysine; for example about 7% by weight of lysine which greatly improves the nutritional quality of the protein source.

In one particular embodiment, the proteins of the composition are hydrolysed, fully hydrolysed or partially hydrolysed. The degree of hydrolysis (DH) of the protein can be between 2 and 20, or between 8 and 40, or between 20 and 60 or between 20 and 80 or more than 10, 20, 40, 60, 80 or 90.

At least 70%, 80%, 85%, 90%, 95% or 97% of the proteins may be hydrolysed. In a particular embodiment, 100% of the proteins are hydrolysed.

In one particular embodiment, the proteins of the composition are plant based protein.

### Carbohydrate

A nutritional composition according to the present invention may contain a carbohydrate source. The carbohydrate may be in an amount of from about 5 g to about 20 g per 100 kcal, or about 10 g to about 15 g per 100 kcal.

Any carbohydrate source conventionally found in nutritional compositions such as lactose, sucrose, saccharose, maltodextrin, starch and mixtures thereof may be used although one of the preferred sources of carbohydrates is lactose.

### Lipid

A nutritional composition according to the present invention may contain lipids and essential fatty acids. The lipids may be in an amount of from about 1 g to about 10 g per 100 kcal, or about 2 g to about 6 g per 100 kcal.

Non-limiting examples of lipids include: palm olein, high oleic sunflower oil, high oleic safflower oil, canola oil, fish oil, coconut oil, bovine milk fat, and combinations thereof. It may be particularly beneficial if the composition comprises fat in an amount of about 25 to about 30 g/100 g dry weight of the composition. Non-limiting examples of essential fatty acids include: linoleic acid (LA), α-linolenic acid (ALA). Compositions of the invention may further contain gangliosides monosialoganglioside-3 (GM3) and disialogangliosides 3 (GD3), and combinations thereof.

### Other Components

A nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin B1, vitamin B2, vitamin B3, vitamin B6, vitamin B12, vitamin E, vitamin C, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population. If necessary, a nutritional composition of the invention may contain emulsifiers and stabilisers such as soy, lecithin, citric acid esters of mono- and diglycerides, and the like.

A nutritional composition of the invention may also contain other substances which may have a beneficial effect, especially on bone health or bone development, such as lactoferrin, osteopontin, TGFbeta, slgA, glutamine, nucleotides, nucleosides, and the like.

### Preparation of Compositions

The compositions according to the present invention may be prepared by any known or otherwise suitable manner. For example, a nutritional composition may be proposed by blending together a source of protein with a carbohydrate source and a lipid source in appropriate proportions. If used, emulsifiers may be included at this stage. Vitamins and minerals may be added at this stage, but may also be added later to avoid thermal degradation. Water, preferably water which has been subjected to reverse osmosis or deionized water, may then be added and mixed in to form a liquid mixture. The temperature of mixing is preferably room temperature, but may also be higher. The liquid mixture may then be thermally treated to reduce bacterial loads. The mixture may then be homogenized.

If it is desired to produce a powdered composition, the homogenized mixture is dried in a suitable drying apparatus, such as a spray drier or freeze drier and converted into powder.

Processes used in the manufacture of formula are based on the concept that the products must be nutritionally adequate and microbiologically safe to consume. Thus, steps that eliminate or restrict microbiological growth are central to production processes. The processing technology for each specific formula is proprietary to the manufacturer but, in general, it involves the preservation of an oil-in-water (o/w) emulsion by dehydration in the case of powder products or, sterilization in the case of ready-to-feed or concentrated liquid products. Powdered formula may be produced using various processes, such as dry blending dehydrated ingredients to constitute a uniform formula or hydrating and wet-mixing a mixture of macro-ingredients, such as fat, protein and carbohydrate ingredients and then evaporating and spray drying the resultant mixture. A combination of the two processes described above may be used where a base powder is first produced by wet-mixing and spray drying all or some of the macro-ingredients and then dry blending the remaining ingredients, including carbohydrate, minerals and vitamins and other micronutrients, to create a final formula. Liquid formulae are available in a ready-to-feed format or as a concentrated liquid, which requires dilution, normally 1:1, with water. The manufacturing processes used for these products are similar to those used in the manufacture of recombined milk.

If it is desired to produce a liquid formula, the homogenized mixture may be filled into suitable containers, preferably aseptically. However, the liquid composition may also be retorted in the container, suitable apparatus for carrying out the filling and retorting of this nature is commercially available.

### Subject

The subject may be any suitable subject. Suitably, the subject may be a mammal. In preferred embodiments, the subject is a human. In other embodiments, the subject is an animal, preferably wherein the animal is a pet. A pet may be an animal selected from dogs, cats, birds, fish, rodents such as mice, rats, and guinea pigs, rabbits, etc. In some embodiments, the pet is a dog. In some embodiments, the pet is a small dog breed.

In some embodiments, the subject is a juvenile. The term “juvenile” may refer to an individual that has not yet reached adulthood. In some embodiments, the subject is an adolescent or a child. The term “adolescent” may refer to an individual during the period from the onset of puberty to adulthood. The term “child” may refer an individual between the stages of birth and puberty.

In some embodiments, the subject is an infant, a toddler, or a young child. The term “infant” may refer to a subject aged from about 0 years to about 1 year. The term “toddler” may refer to a subject aged from about 1 year to about 3 years. The term “young child” may refer to a subject aged from about 3 years to about 10 years, from about 3 years to about 9 years, from about 3 years to about 8 years, from about 3 years to about 7 years, from about 3 years to about 6 years, or from about 3 years to about 5 years.

In some embodiments, the subject is a newborn infant. Newborn infants typically have low vitamin K and may be at risk of vitamin K deficiency bleeding (see e.g. Araki, S. and Shirahata, A., 2020. Nutrients, 12 (3), p. 780). In some embodiments, the subject is a neonate.

In some embodiments, the subject is aged about 5 years or less, about 4 years or less, about 3 years or less, about 2 years or less, or about 1 year or less. In some embodiments, the subject is aged about 12 months or less, about 11 months or less, about 10 months or less, about 9 months or less, about 8 months or less, about 7 months or less, or about 6 months or less. In some embodiments, the subject is aged about 0.5 months or older. In some embodiments, the subject is aged from about 0.5 months to about 6 months.

In some embodiments, the subject is an adolescent or an adult. In some embodiments, the subject is an adult. In some embodiments, the subject is elderly. In some embodiments, the subject is at least 60 years of age, at least 65 years of age, at least 70 years of age, at least 75 years of age, or at least 80 years of age.

The subject may have or may be at risk of vitamin K2 deficiency. The subject may have or may be at risk of primary vitamin K2 deficiency, i.e. insufficient consumption of vitamin K2. The subject may have or may be at risk of secondary vitamin K2 deficiency. Secondary deficiencies can occur in people who consume adequate amounts, but have malabsorption conditions, such as cystic fibrosis or chronic pancreatitis, and in people who have liver damage or disease. Secondary vitamin K deficiency can also occur in people who have a prescription for a vitamin K antagonist drug. Long-term antibiotic use and long-term glucocorticoid use can also affect vitamin K levels, particularly in children (see e.g. Kozioł-Kozakowska, A. and Maresz, K., 2022. Children, 9 (1), p. 78).

In some embodiments, the subject has or is at risk of dyslipidemia, diabetes, thalassemia major (TM), cystic fibrosis (CF), inflammatory bowel diseases (IBD), or liver diseases.

In some embodiments, the subject has cystic fibrosis, inflammatory bowel disease (e.g. Crohn's Disease or ulcerative colitis), liver disease (e.g. chronic liver disease), long-term antibiotic use, or long-term glucocorticoid use.

The subject may have or may be at risk of reduced bone growth and/or bone strength. Vitamin K is required for the gamma-carboxylation of osteocalcin in bone and may be required to form strong bones (see e.g. Hamidi, M. S., et al., Journal of clinical densitometry, 16 (4), pp. 409-413).

The present invention is suitable for children who were born preterm or with low-birth weight or experienced intra-uterine growth retardation or who suffered from growth stunting because of malnutrition or experienced disease such as Crohn's disease and/or celiac disease and/or cancer or who were treated with drugs leading to malabsorption, anorexia and/or metabolic bone disease, such as chemotherapy drugs and/or corticosteroids. In some embodiments, the subject was born preterm or with low-birth weight or experienced intra-uterine growth retardation, or with intra-uterine malnutrition or suffered growth delay. The present invention is also suitable for subjects at risk of bone disease, having a family history of bone disease, or having already experienced at least one, preferably several, episode(s) of fracture.

In some embodiments, the subject suffered from and/or is suffering from stunted growth. The definition of stunting may refer to the “height for age” value to be less than two standard deviations of the WHO Child Growth Standards median (see e.g. De Onis, M. and Branca, F., 2016. Maternal & child nutrition, 12, pp. 12-26).

In some embodiments, the subject suffered from and/or is suffering from faltering growth. The term “faltering growth” may describe a pattern of slower weight gain than expected for age and sex in children and other adolescents (see e.g. King, C. and Davis, T., 2010. European journal of clinical nutrition, 64 (1), pp. S11-S13). In some embodiments, the subject suffered from and/or is suffering from growth stunting and/or faltering growth because of malnutrition or experienced disease such as anorexia, Crohn's disease and/or celiac disease. In some embodiments, the subject suffered from and/or is suffering from growth stunting and/or faltering growth because of treatment with drugs leading to malabsorption, anorexia and/or metabolic bone disease, such as chemotherapy drugs and/or corticosteroids.

In some embodiments, the subject was born preterm or with low-birth weight or experienced intra-uterine growth retardation. The term “preterm infant” may refer to an infant born at least than 37 weeks gestational age. The term “low birth weight infant” may refer to an infant having a live-born weight less than 2,500 g.

Possible factors inducing vitamin K deficiency in newborns include poor placental transfer of vitamin, immature gut flora, low vitamin K content in breast milk and substantial differences among individuals, poor intestinal absorption of vitamin K, and low activity level of vitamin K epoxide reductase (see e.g. Araki, S. and Shirahata, A., 2020. Nutrients, 12 (3), p. 780).

Methods for Treating and/or Preventing Vitamin K2 Deficiency

The prebiotic agent (or combination therewith) may be used to treat and/or prevent vitamin K2 deficiency.

In one aspect, the present invention provides a prebiotic agent for use in treating and/or preventing vitamin K2 deficiency in a subject.

In another aspect, the present invention provides use of a prebiotic agent in the manufacture of a medicament for treating and/or preventing vitamin K2 deficiency in a subject.

In another aspect, the present invention provides a method for treating and/or preventing vitamin K2 deficiency in a subject in need thereof, the method comprising administering to the subject an effective amount of a prebiotic agent.

The present inventors have surprisingly found that vitamin K2 production in the gastrointestinal tract (e.g. via conversion of vitamin K1) is promoted by a prebiotic agent, for example comprising bovine milk oligosaccharides (BMOs), human milk oligosaccharides (HMOs), and/or cello-oligosaccharides (COS). The prebiotic agent (or a combination therewith) may treat and/or prevent vitamin K2 deficiency by promoting vitamin K2 production in the subject's gut. For example, the prebiotic agent may treat and/or prevent vitamin K2 deficiency by promoting de novo menaquinone-7 production and/or bioconversion from phylloquinone to menaquinone-4 in the subject's gut.

A vitamin K2 deficiency may be diagnosed by any suitable method known in the art. For example, a vitamin K deficiency may lead to reduced blood clotting, and in severe cases, can result in reduced clotting, increased bleeding, and increased prothrombin time. The diagnosis of vitamin K deficiency bleeding is commonly indicated by a prolonged activated partial thromboplastin time (APTT) and prothrombin time (PT), e.g. a PT international normalized ratio (INR)≥4 or a value>4 times the normal values in the presence of normal platelet count and fibrinogen level (see e.g. Araki, S. and Shirahata, A., 2020. Nutrients, 12(3), p. 780).

Methods for enhancing bone growth and/or bone strength or preventing bone loss and decreased bone strength. Treating and/or preventing vitamin K2 deficiency in a subject, may thereby enhance bone growth and/or bone strength or prevent bone loss and decreased bone strength in the subject (see e.g. Hamidi, M. S., et al., Journal of clinical densitometry, 16 (4), pp. 409-413).

Within the context of the present invention, the term “enhancing bone growth and/or bone strength, or limiting/preventing bone loss” may refer to, in particular, one or more of the following physiological processes: bone catch-up growth, bone mass acquisition, optimization of peak bone mass, promotion of bone formation, promotion of bone anabolism, promotion of bone mineralization, increase of bone mineral density and micro-architecture, modulation of bone biomechanical properties, and modulation the ratio of bone formation and/or bone resorption, bone mass maintenance, reduction of bone resorption

As used herein “bone quality” may refer to aspects of bone composition and structure that contribute to bone strength independently of bone mineral density. These include bone turnover, microarchitecture, mineralisation, microdamage and the composition of bone matrix and mineral. Methods to measure bone quality are known in the art.

As used herein, “promoting bone growth and/or strength” may refer to the support of normal bone growth and/or strength, for example during childhood and adolescence. Supporting normal bone growth and/or strength may result in normal bone anatomy and physiology. Suitable methods and parameters to determine bone growth and bone strength will be known to the skilled person (see e.g. Donnelly, E., 2011. Clinical Orthopaedics and Related Research, 469(8), pp. 2128-2138). Suitably, normal bone growth and/or strength may be determined using one or more bone parameter selected from: trabecular bone volume fraction (BV/TV), bone mineral density (BMD), bone mineral content (BMC), cortical bone volume (Ct. BV), medio-lateral diameter, antero-posterior diameter, bone ultimate force (FMax), and bone stiffness. In some embodiments, normal bone growth and/or strength is determined using one or more bone parameter selected from: bone mineral density (BMD), trabecular bone volume fraction (BV/TV), cortical bone volume (Ct. BV), and bone ultimate force (FMax). Suitable methods to determine these parameters will be available to the skilled person.

A regular nutritional supply of the composition according to the present invention is also useful for preventing the bone loss that occurs with ageing and/or to protect bone cells during bone aging.

In an embodiment, the said composition is for use i) improving bone quality; ii) preventing or treating disorders linked to an imbalance in the relationship between bone formation and bone resorption.

By “reduction/inhibition of bone resorption” is meant according to the invention inhibition of the destructive activity of bone tissue by the osteoclast cells. In order to verify that the supply of the composition in man or animals inhibits bone resorption, the specialist skilled in the art can measure the urinary excretion of desoxypyridinoline as described in the examples, a diminution of the expression of desoxypyridinoline being the reflection of inhibition of bone resorption.

The supply of the composition according to the invention to an animal organism induces simultaneously a stimulation of bone formation and inhibition of bone resorption, the overall increase of bone mineralisation, and hence of the bone density, being the result of the induction of these two mechanisms.

In order to determine whether a subject presents a state of reduced bone mass and as a consequence requires a supply of the composition according to the present invention, the specialist skilled in the art will be able to refer in particular to the report of the World Health Organisation (WHO) of 1994 entitled “Assessment of fracture risk and its application to screening for post-menopausal osteoporosis” (WHO Technical Series—843).

The composition according to the present invention is also designed for Individuals presenting symptoms of bone deficit or likely to suffer from bone deficit, i.e. from an imbalance in the relationship between bone formation and bone resorption which, if it continues, induces a diminution of the bone mass. A composition according to the invention is also designed for individuals presenting symptoms of bone deficit resulting from a fracture, an operation or also a dental disease.

In particular, the composition is designed to prevent or treat diseases selected from osteoporosis, Paget's disease, bone loss or osteolysis observed close to a prosthesis, metastatic bone diseases, hypercalcemia due to a cancer, multiple myelomas, periodontal diseases or osteoarthritis.

As has already been mentioned above, many disorders linked to an imbalance of bone metabolism, such as osteoporosis, develop gradually over a long period of time and require chronic treatments. Their prevention or their treatment can hence be carried out by means of a regular supply of the composition according to the present invention, preferably in the form of a nutritional composition.

Similarly, a regular nutritional supply of the composition according to the present invention to individuals, humans or animals, is such as to make possible the production of a high bone density and an elevated peak bone mass by stimulation of bone formation when these individuals attain adult age.

A regular nutritional supply of the composition according to the present invention is also useful for preventing the bone loss that occurs with ageing (that may lead to osteoporosis) and/or to protect bone cells during bone aging.

Thus, in man and other mammals a great variety of disorders are related to abnormal metabolism of bone resorption and bone formation, leading to an imbalance in metabolism or bone remodelling.

Of the pathological disorders related to an imbalance in bone metabolism, particular mention may be made of the disorders or diseases such as osteoporosis, Paget's disease, bone loss or osteolysis observed close to a prosthesis, metastatic bone diseases, hypercalcemia due to a cancer, multiple myelomas and periodontal diseases. Some of the disorders or diseases of bone metabolism may be caused by long-term immobilisation, for example long-term hospitalisation or even after a period of weightlessness. Of the disorders linked to abnormal bone resorption, the most common is osteoporosis, the most frequent manifestation of which is observed in women after the start of menopause. Osteoporosis is a systemic skeletal disease characterised by a reduction of the bone mass and a deterioration of the microarchitecture of bone tissue, associated with an increase of the fragility of the bone and its susceptibility to fracture.

Since osteoporosis, like other disorders associated with bone loss, constitutes a chronic disorder, its prevention and its treatment must be planned in the long term.

It is currently accepted that early treatment must be preferred because the two critical phases for bone capital are: the period of growth during which the maximal bone mass (peak bone mass) is acquired; ageing which conditions the rate of loss of bone mass. The prevention of osteoporosis must hence no longer be restricted to the elderly individual.

Moreover, in man and animals, there are many conditions characterised by the need to increase bone formation. For example, in the case of bone fractures, it is necessary to stimulate bone growth in order to accelerate complete repair of the bone. This need is also present in the periodontal diseases, the metastatic diseases of bone, the osteolytic diseases and the conditions under which repair of the connective tissue is required, for example for the cicatrisation or regeneration of defects or traumatisms of cartilage. The stimulation of bone growth is also required in the case of primary and secondary hyperparathyroidism, as well as in osteoporosis associated with diabetes and in osteoporosis associated with glucocorticoids.

### Methods for Promoting Catch-Up Growth

Treating and/or preventing vitamin K2 deficiency in a subject, may thereby promote catch-up growth.

As used herein, “catch-up growth” may refer to height velocity above the limits of normal for age for at least 1 year after a transient period of growth inhibition and may be complete or incomplete (see e.g. Wit, J. M. and Boersma, B., 2002. Journal of Pediatric Endocrinology and Metabolism, 15, pp. 1229-1242.

Suitable method and parameters to determine catch-up growth will be known to the skilled person. Suitably, catch-up growth may be determined using height velocity or height standard deviation score (see e.g. Frongillo, E. A., Leroy, J. L. and Lapping, K., 2019. Advances in Nutrition, 10 (3), pp. 372-379 and Desmond, C. and Casale, D., 2017. PloS one, 12 (12), p.e0189135).

In some embodiments, the catch-up growth is determined in absolute terms of linear growth (i.e. the height deficit from the healthy reference population mean is reduced). In some embodiments, the catch-up growth is determined in relative terms of linear growth (i.e. the height-for-age z-score is improved and/or passes the −2SD or −1SD cut-off points).

### EXAMPLES

The invention is further described with reference to the following examples. It will be appreciated that the invention as claimed is not intended to be limited in any way by these examples.

### Example 1—Promoting Vitamin K2 Production in the Gastrointestinal Tract

To evaluate intestinal production of vitamin K2, a Simulator of the Human Microbial Ecosystem (SHIMER) was used (see Van de Wiele, T., et al., 2015. The Impact of Food Bioactives on Health: in vitro and ex vivo models, pp. 305-317). SHIME assays typically consist of a colonic fermentation of a selected dose of the test compound(s) under simulated conditions representative for the gastrointestinal tract of a subject. Production of menaquinone-7 was determined by supercritical fluid chromatography tandem mass spectrometry (SFC-MS/MS).

For the current set of experiments a two-stage batch system mimicking the upper gastro-intestinal tract (Upper GIT, stomach and small intestine) and colonic conditions were used as simplified SHIME® system. A cow milk-based infant beverage, also called infant formula-milk, containing minerals adapted for the age group was used in these studies.

In order to simulate the absorptive processes occurring in the small intestine of infants, a dialysis approach was applied by using a cellulose membrane with a cut-off of 14 kDa. By introducing the small intestinal suspension within a dialysis membrane, molecules such as digested amino acids, sugars, micronutrients and minerals were gradually removed from the upper gastro-intestinal matrices.

Furthermore, a gradual pH decrease during the stomach incubation going from 5.5 to 3.0 during 1 hour of incubation was implemented to simulate the gastric pH of infants. Also, during the first 30 minutes of small intestinal incubation (duodenum), a fixed pH of 4.5 was implemented to allow the available minerals to optimally absorb. In the following 145 minutes of the small intestinal phase (jejunum+ileum), a pH of 7 was introduced. The milk matrix after exposure to gastric and small intestinal conditions was transferred to the colonic compartment containing the fecal sample of an infant.

Fresh fecal material was collected from a 12-month-old infant donor. Fecal suspension was prepared and mixed with a protectant. At the start of the short-term colonic incubation, the test ingredients (see below) were added to sugar-depleted nutritional medium containing basal nutrients present in the colon (e.g., host-derived glycans such as mucin).

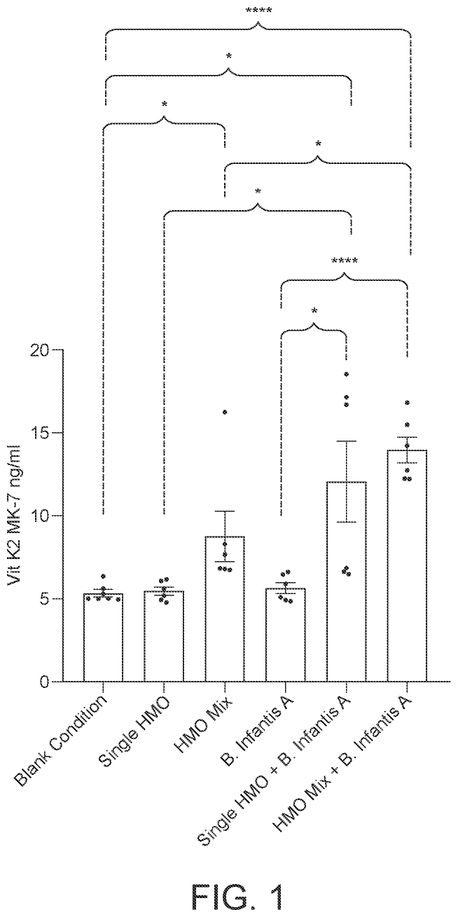

In a first set of experiments, the following groups (test ingredients) were evaluated: Blank; Single HMO (1.3 g/L 2FL); HMO mix (2.5 g/L 2FL, DiFL, LNnT, LNT, and 6SL); B. infantis (10⁷ cfu/ml); Single HMO+B. infantis; HMO mix+B. infantis. The results are shown in FIG. 1. The HMO mix increased menaquinone-7 production by 65% compared to the negative control. The addition of B. infantis further increased menaquinone-7 production by 163% compared to the negative control.

In a second set of experiments, the following groups (test ingredients) were evaluated (in the absence of milk matrix): Blank; and HMOs+BMOs (7.2 g/L total). The composition of HMOs and BMOs is shown in the table below:

| | g/L | % of total |
|---|---|---|
| 2FL | 0.252 | 3.50 |
| DiFL | 0.036 | 0.50 |
| LNnT | 0.126 | 1.75 |
| LNT | 0.087 | 1.21 |
| 6SL | 0.049 | 0.68 |
| 3SL | 0.046 | 0.64 |
| BMOs | 6.602 | 91.71 |
| Total | 7.199 | 100 |

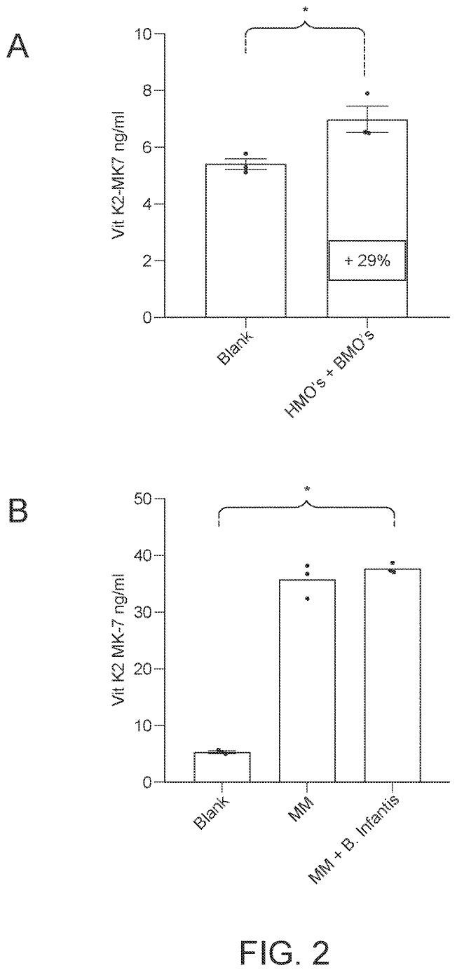

The results are shown in FIG. 2A. The mixture of BMOs and HMOs increase production of menaquinone-7 by 29% compared to the negative control. It was further shown that a milk matrix comprising a mixture of six HMOs significantly increases production of menaquinone-7 and that the addition of B. infantis further increased production (see FIG. 2B).

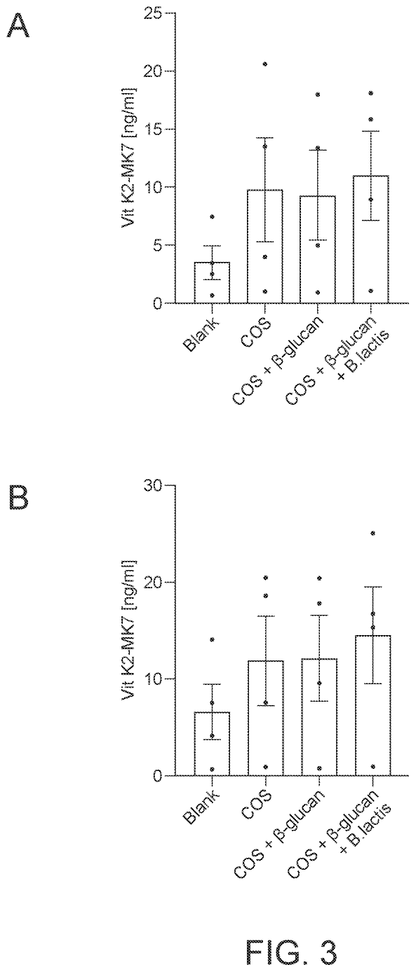

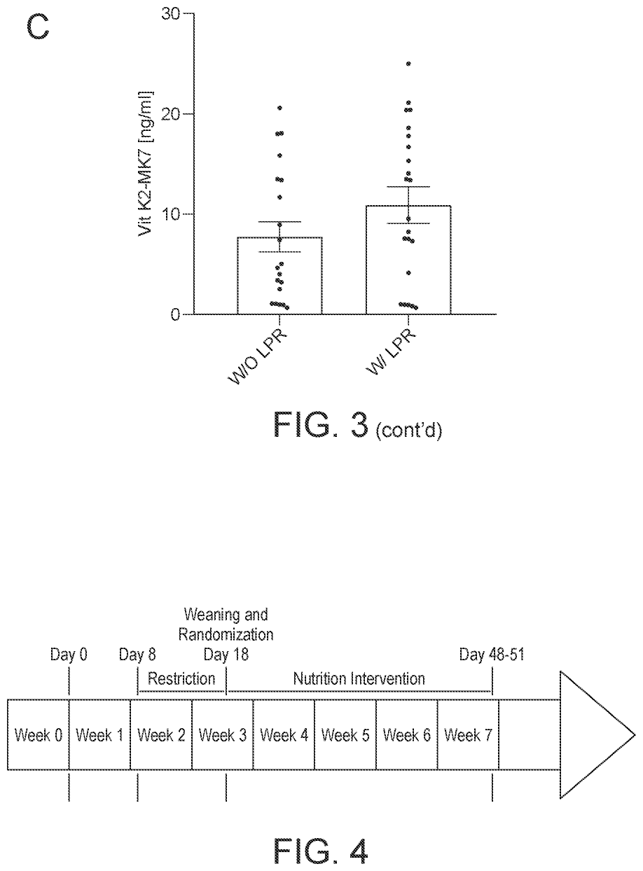

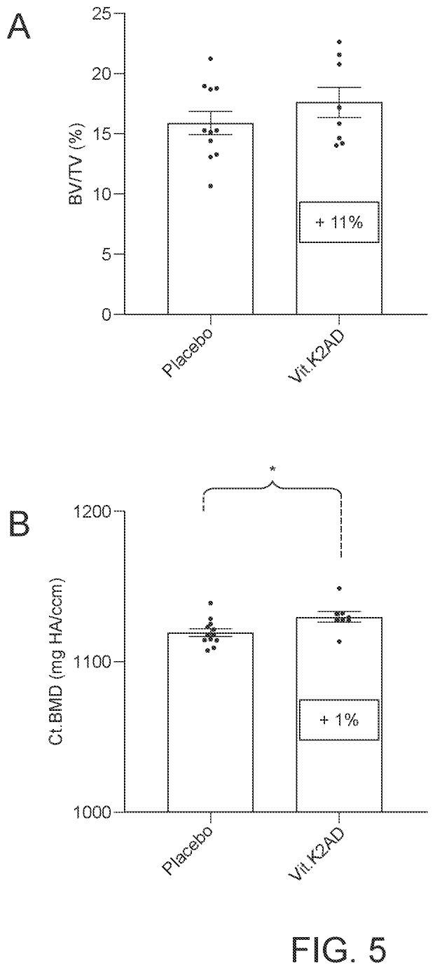

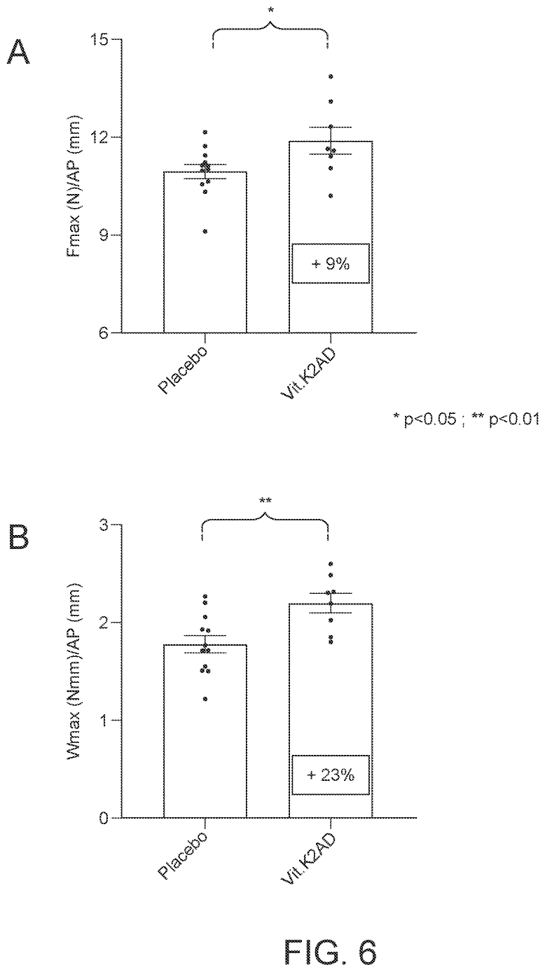

In a final set of experiments, the following groups were evaluated (in a milk matrix in the absence or presence of L. rhamnosus LPR): Blank; COS (2700 mg/L); COS (2025 mg/L)+β-glucan (675 mg/L); and COS (2025 mg/L)+β-glucan (675 mg/L)+B. lactis (4.5×10⁶ cfu/ml). The results, in the absence or presence of L. rhamnosus LPR, are shown in FIGS. 3A and 3B, respectively. In the absence of LPR, COS increased the production of MK-7 by 178%. In the presence of LPR, the addition of B. lactis further increase the production of MK-7 by 22%.

Further, when all groups were combined, it was shown that the addition of Lactobacillus rhamnosus LPR (4.5×10⁷ cfu/ml) can further increase vitamin K2 production in the gastrointestinal tract by about 40% (see FIG. 3C).

### Example 2—Preclinical Experimental Results

A preclinical experimental setup was followed as shown in FIG. 4. The number of pups per BALB/c mother was increased by 50% from D8 to D18 to induce a food restriction. In both groups (normal and faltering growth), weaning happened at D18. Male and female mice then received food ad libitum and a daily nutritional supplementation through pipet feeding for 30 days.

As can be seen from FIGS. 5A&B, vitamin K2AD promoted bone quality by improving trabecular bone volume (BV/TV ratio) (trend increase) and cortical bone mineral density (Ct.BMD) (significant increase).

Additionally, FIGS. 6A&B demonstrates that vitamin K2AD enhanced bone strength by increasing both the force (Fmax) and the energy (Wmax) necessary to reach the point of failure of femurs.

### EMBODIMENTS

Various preferred features and embodiments of the present invention will now be described with reference to the following numbered paragraphs (paras).
1. A prebiotic agent for use in treating and/or preventing vitamin K2 deficiency in a subject.
2. The prebiotic agent for use according to any preceding para, wherein the prebiotic agent is administered in combination with vitamin K1.
3. The prebiotic agent for use according to para 2, wherein the prebiotic agent and vitamin K1 are administered separately, simultaneously or sequentially, preferably wherein the prebiotic agent and vitamin K1 are administered simultaneously.
4. The prebiotic agent for use according to any preceding para, wherein the prebiotic agent is administered in combination with a probiotic agent.
5. The prebiotic agent for use according to para 4, wherein the prebiotic agent and the probiotic agent are administered separately, simultaneously or sequentially, preferably wherein the prebiotic agent and the probiotic agent are administered simultaneously.
6. The prebiotic agent for use according to para 4 or 5, wherein the probiotic agent comprises Lactobacillus rhamnosus, Bifidobacterium infantis and/or Bifidobacterium lactisi.
7. The prebiotic agent for use according to any preceding para, wherein the prebiotic agent is selected from one or more of: bovine milk oligosaccharides (BMOs), human milk oligosaccharides (HMOs), cellobiose, cello-oligosaccharides (COS), inulin, lactose, fructo-oligosaccharides (FOS), galacto-oligosaccharides (GOS), and β-glucan.
8. The prebiotic agent for use according to any preceding para, wherein the prebiotic agent is selected from one or more of: bovine milk oligosaccharides (BMOs), a human milk oligosaccharide (HMO) mixture, and cello-oligosaccharides (COS).
9. The prebiotic agent for use according to any preceding para, wherein the prebiotic agent comprises BMOs in an amount of from about 80 wt % to about 100 wt %, with respect to the total weight of the prebiotic agent.
10. The prebiotic agent for use according to any preceding para, wherein the prebiotic agent comprises or consists of at least one sialylated oligosaccharide, at least one fucosylated oligosaccharide, and/or at least one N-acetylated oligosaccharide.
11. The prebiotic agent for use according to para 10, wherein the at least one sialylated oligosaccharide is selected from the group consisting of 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof, preferably wherein the at least one sialylated oligosaccharide is selected from 3′-sialyllactose (3′-SL), 6′-sialyllactose (6′-SL) and combinations thereof.
12. The prebiotic agent for use according to para 10 or 11, wherein the at least one fucosylated oligosaccharide is selected from the group consisting of 2′-fucosyllactose (2′FL), 3-fucosyllactose (3FL), difucosyllactose (diFL), lacto-N-fucopentaose-I (LNFP-I), lacto-N-fucopentaose-II (LNFP-II), lacto-N-fucopentaose-III (LNFP-III), lacto-N-fucopentaose-V (LNFP-V), lacto-neofucopentaose V (LNnFP-V), lacto-N-difucosylhexaose-I (LNDFH-1), lacto-N-neodifucosylhexaose (LNnDFH), monofucosyllacto-n-hexaose-III (MFNLH-III), difucosyllacto-N-hexaose-a (DFLNHa) and combinations thereof, preferably wherein the at least one fucosylated oligosaccharide is 2′-fucosyllactose (2′FL) and/or difucosyllactose (diFL).
13. The prebiotic agent for use according to any of paras 10 to 12, wherein the at least one N-acetylated oligosaccharide is selected from the group consisting of N-acetyl-glucosamine, N-acetyl-galactosamines, lacto-N-tetraose (LNT), lacto-N-neotetraose (LNnT), and combinations thereof, preferably wherein the at least one N-acetylated oligosaccharide is selected from lacto-N-tetraose (LNT), lacto-N-neotetraose (LNnT) and combinations thereof.
14. The prebiotic agent for use according to any preceding para, wherein the prebiotic agent comprises:
   (a) from about 0.5 wt % to about 2 wt % with respect to the total weight of the oligosaccharide mixture, of at least one sialylated oligosaccharide;
   (b) from about 2 wt % to about 6 wt %, with respect to the total weight of the oligosaccharide mixture, of at least one fucosylated oligosaccharide; and/or
   (c) from about 1 wt % to about 4 wt %, with respect to the total weight of the oligosaccharide mixture, of at least one N-acetylated oligosaccharide.
15. The prebiotic agent for use according to any preceding para, wherein the prebiotic agent comprises or consists of cello-oligosaccharides (COS).
16. The prebiotic agent for use according to any preceding para, wherein the prebiotic agent is administered to the subject in a total amount of from about 0.5 g/day to about 10 g/day.
17. The prebiotic agent for use according to any preceding para, wherein vitamin K1 is administered to the subject in an amount of from about 5 μg/day to about 100 μg/day.
18. The prebiotic agent for use according to any preceding para, wherein a probiotic agent is administered to the subject in a total amount of from about 10⁶ cfu/day to about 10¹² cfu/day.
19. The prebiotic agent for use according to any preceding para, wherein the prebiotic agent is provided in the form of a nutritional composition.
20. The prebiotic agent for use according to any preceding para, wherein the prebiotic agent is provided in the form of a medical food product for clinical nutrition.
21. The prebiotic agent for use according to para 19 or 20, wherein the composition comprises the prebiotic agent in a total amount of from about 0.5 g/100 g to about 10 g/100 g, on a dry weight basis.
22. The prebiotic agent for use according to any of paras 19 to 21, wherein the composition comprises vitamin K1 in an amount of about 5 μg/100 g to about 100 μg/100 g, on a dry weight basis.
23. The prebiotic agent for use according to any of paras 19 to 22, wherein the composition comprises a probiotic agent in an amount of about 10⁶ cfu/100 g to about 10¹² cfu/100 g, on a dry weight basis.
24. The prebiotic agent for use according to any preceding para, wherein the subject is an infant, a toddler, or a child.
25. The prebiotic agent for use according to any preceding para, wherein the subject has or is at risk of vitamin K2 deficiency.
26. The prebiotic agent for use according to any preceding para, wherein the subject has or is at risk of dyslipidemia, diabetes, thalassemia major (TM), cystic fibrosis (CF), inflammatory bowel diseases (IBD), or chronic liver diseases.
27. The prebiotic agent for use according to any preceding para, wherein the subject has or is at risk of reduced bone growth and/or bone strength.
28. The prebiotic agent for use according to any preceding para, wherein the prebiotic agent treats and/or prevents vitamin K2 deficiency by promoting vitamin K2 production in the subject's gut.
29. The prebiotic agent for use according to any preceding para, wherein the prebiotic agent treats and/or prevents vitamin K2 deficiency by promoting de novo menaquinone-7 production and/or bioconversion from phylloquinone to menaquinone-4 in the subject's gut.
30. Use of a prebiotic agent in the manufacture of a medicament for treating and/or preventing vitamin K2 deficiency in a subject.
31. A method for treating and/or preventing vitamin K2 deficiency in a subject in need thereof, the method comprising administering to the subject an effective amount of a prebiotic agent.
32. Use of a prebiotic agent to promote vitamin K2 production in the gut of a subject.
33. The use according to para 32, wherein the prebiotic agent promotes de novo menaquinone-7 production and/or bioconversion from phylloquinone to menaquinone-4 in the subject's gut.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A method of treating and/or preventing vitamin K2 deficiency in a subject, the method comprising administering a prebiotic agent to the subject.

2. The method according to claim 1, further comprising administering a vitamin mixture to promote vitamin K2 production in the gut of a subject.

3. The method according to claim 2, wherein the vitamin mixture comprises vitamin K2, A and/or D.

4. The method according to claim 1, wherein the prebiotic agent is administered in combination with vitamin K1.

5. The method according to claim 1, wherein the prebiotic agent is administered in combination with a probiotic agent.

6. The method according to claim 5, wherein the probiotic agent comprises Lactobacillus rhamnosus, Bifidobacterium infantis and/or Bifidobacterium lactis.

7. The method according to claim 1, wherein the prebiotic agent is one or more of: bovine milk oligosaccharides (BMOs), human milk oligosaccharides (HMOs), cellobiose, cello-oligosaccharides (COS), fructo-oligosaccharides (FOS), galacto-oligosaccharides (GOS), and β-glucan.

8. The method according to claim 1, wherein the prebiotic agent comprises BMOs in an amount from about 80 wt % to about 100 wt %, with respect to the total weight of the prebiotic agent.

9. The method according to claim 1, wherein the prebiotic agent comprises HMOs.

10. The method according to claim 1, wherein the prebiotic agent comprises COS.

11. The method according to claim 1, wherein the prebiotic agent is administered to the subject in a total amount from about 0.5 g/day to about 10 g/day.

12. The method according to claim 4, wherein the vitamin K1 is administered to the subject in an amount from about 5 μg/day to about 100 μg/day, and/or wherein the probiotic agent is administered to the subject in a total amount from about 10⁶ cfu/day to about 10¹² cfu/day.

13. The method according to claim 1, wherein the prebiotic agent is administered in a nutritional composition.

14. The method according to claim 1, wherein the subject is an infant, a toddler, or a child.

15. The method according to claim 1, wherein the subject has or is at risk of dyslipidemia, diabetes, thalassemia major (TM), cystic fibrosis (CF), inflammatory bowel diseases (IBD), or chronic liver diseases, and/or wherein the subject has or is at risk of reduced bone growth and/or bone strength.

16. The method according to claim 1, wherein the prebiotic agent is administered in an amount effective to enhance bone growth, bone mineralisation and/or bone strength or limit or prevent bone loss and strength in the subject.

17. The method according to claim 1, wherein the prebiotic agent is administered in an amount effective to treat and/or prevent vitamin K2 deficiency by promoting vitamin K2 production in the subject's gut.

18. (canceled)

19. The method according to claim 1, wherein the prebiotic agent is selected from one or more of: bovine milk oligosaccharides (BMOs), a human milk oligosaccharide (HMO) mixture, and cello-oligosaccharides (COS).

20. The method according to claim 1, wherein the prebiotic agent comprises at least one sialylated oligosaccharide, at least one fucosylated oligosaccharide, and/or at least one N-acetylated oligosaccharide.
